(19) United States
(12) Patent Application Publication
(10) Pub. No.: US 2026/0294776 A1
(43) Pub. Date: Oct. 1, 2026

(54) COSMETIC SYSTEMS FOR STRUCTURAL MODIFICATION OF HAIR FIBERS USING LIGNIN STARCH NANOPARTICLES

(71) Applicant: Rose Lenore Bonner, Jamestown, NC (US)

(72) Inventor: Rose Lenore Bonner, Jamestown, NC (US)

(21) Appl. No.: 19/547,865

(22) Filed: Feb. 24, 2026

Related U.S. Application Data
(60) Provisional application No. 63/762,457, filed on Feb. 24, 2025.

(51) Int. Cl. A61K 8/66; A45D 7/04; A61K 8/73; A61K 8/97; A61Q 5/06

(52) U.S. Cl. CPC A61K 8/66; A45D 7/04; A61K 8/732; A61K 8/97; A61Q 5/06; A45D 2200/25; A61K 2800/884

(57) ABSTRACT

A hair treatment composition is disclosed comprising composite nanoparticles including a lignin phase and a starch phase, an enzyme capable of catalyzing oxidative crosslink formation of lignin phenolic moieties, and a cosmetically acceptable carrier. The composite nanoparticles penetrate cuticle interstices and localize within a cortex region of a hair fiber. Following cortical localization, enzyme activation induces polymerization of the lignin phase to form a polymerized intrafiber scaffold that is physically distinct from keratin and that forms substantially without cleavage of disulfide bonds. The intrafiber scaffold reinforces internal hair fiber structure and improves mechanical properties of the hair. In certain embodiments, chromogenic stabilizers are included to mitigate discoloration associated with enzymatic oxidation. The compositions and methods provide internal reinforcement of hair fibers without chemically altering keratin or disrupting cuticle integrity.

This application claims priority to U.S. Provisional Application No. 63/762,457, filed Feb. 24, 2025, entitled “Nanoparticles of Buckyballs in Hair,” the entire contents of which are incorporated herein by reference.

### FEDERALLY SPONSORED RESEARCH AND DEVELOPMENT

Not applicable

### FIELD OF INVENTION

The present disclosure relates generally to cosmetic and personal care compositions and methods for treating keratinous fibers. More particularly, the disclosure relates to compositions, methods, and treatment systems for modifying the structural and mechanical properties of hair fibers by forming a biodegradable polymeric scaffold within the hair fiber cortex using plant-derived composite nanoparticles and enzyme-activated polymerization/enzyme-mediated polymerization, while maintaining cuticular integrity and avoiding chemical modification of keratin disulfide bonds.

### BACKGROUND OF THE INVENTION

Hair modification and styling technologies have traditionally relied on chemical and thermal processes to alter the shape, texture, strength, or appearance of keratin fibers. Conventional relaxing, straightening, perming, and reshaping treatments typically involve strongly alkaline compositions, reducing agents, oxidizing agents, or combinations thereof that chemically modify disulfide bonds within keratin. While such treatments can produce durable cosmetic effects, they frequently cause cumulative and irreversible damage to the hair fiber, including cuticle lifting, increased porosity, dryness, loss of tensile strength, and increased susceptibility to breakage.

More recent cosmetic technologies have sought to provide alternatives to harsh disulfide chemistry by introducing bond-building agents, crosslinking systems, peptides, or other conditioning chemistries intended to strengthen hair fibers or improve resistance to damage. Certain formulations are designed to penetrate the hair cuticle and deliver reactive components into the cortex, where crosslinking or bonding reactions are intended to occur. Other approaches attempt to form three-dimensional networks within the hair fiber to promote shape retention or mechanical reinforcement. Although these approaches may reduce reliance on traditional relaxer chemistries, they generally involve molecular-scale interactions with keratin proteins, chemical crosslinking of hair components, or incorporation of small reactive molecules that alter the native keratin network.

In parallel, nanoparticle-based cosmetic systems have been explored for delivering active agents into hair fibers. However, existing nanoparticle technologies often function primarily as carriers for dyes, conditioners, or small-molecule actives, rather than as structural elements that form a persistent internal reinforcement architecture within the hair fiber. Moreover, certain nanoparticle systems raise concerns regarding biocompatibility, environmental persistence, or accumulation in biological tissues.

Accordingly, there remains a need for cosmetic hair treatment technologies that provide internal reinforcement and mechanical modification of hair fibers without relying on caustic conditions, disulfide bond cleavage, or direct chemical modification of keratin proteins. There is a further need for such systems to be biocompatible, plant-derived, environmentally responsible, and capable of delivering durable structural effects while preserving cuticle integrity and scalp-compatible pH conditions. The present disclosure addresses these and other needs by providing compositions and methods that introduce a biodegradable polymeric scaffold within the cortex of the hair fiber through enzyme-activated assembly of plant-derived composite nanoparticles.

### SUMMARY OF THE INVENTION

The disclosure also provides support for a hair treatment composition comprising: (a) composite nanoparticles each comprising a carboxyl-functionalized lignin phase and a starch phase configured to self-assemble into spherical nanostructures having average diameters of about 50-200 nm, (b) a laccase enzyme present in an amount effective to catalyze oxidative crosslink formation of carboxyl moieties after intrafiber localization, and (c) a cosmetically acceptable aqueous carrier, wherein the composition has a pH of 5.5-6.0, and forms within the hair cortex a polymerized lignin-based intrafiber scaffold that is physically distinct from keratin and formed without disulfide bond cleavage. In a first example of the system, polymerization forms a percolating nanoscale intrafiber scaffold within the cortex occupying interstitial spaces between keratin microfibrils. In a second example of the system, optionally including the first example, the composite nanoparticles penetrate through a coil-bend inflection region (“Z-entry junction”) exhibiting a localized angular change in the curl path that produces a transient porous junction, facilitating deeper nanoparticle entry, having the capacity to extend across the plurality of keratin microfibrils within the cortex. In a third example of the system, optionally including one or both of the first and second examples, Z-entry penetration reduces tensile stress on the condensed-coil region during elongation by distributing mechanical load away from torsion-dense zones. In a fourth example of the system, optionally including one or more or each of the first through third examples, the composite nanoparticles comprise lignin-rich functional domains stabilized by starch-rich supportive domains within a unified composite morphology, so that the lignin-rich domains serve as the polymerizable phase upon laccase activation. In a fifth example of the system, optionally including one or more or each of the first through fourth examples, the nanoparticles exhibit a zeta potential promoting electrostatic association with hair fibers. In a sixth example of the system, optionally including one or more or each of the first through fifth examples, scaffold density within the cortex is adjustable by nanoparticle concentration. In a seventh example of the system, optionally including one or more or each of the first through sixth examples, the nanoparticles have diameters within 50-150 nm. In a eighth example of the system, optionally including one or more or each of the first through seventh examples, the nanoparticles have diameters within 80-120 nm. In a ninth example of the system, optionally including one or more or each of the first through eighth examples, the lignin phase comprises functionalized lignin derived from a lignin source selected from the group consisting of organosolv lignin, kraft lignin, soda lignin, and combinations thereof, the lignin comprising carboxyl-functional groups. In a tenth example of the system, optionally including one or more or each of the first through ninth examples, the starch phase is derived from a starch source selected from the group consisting of corn starch, potato starch, tapioca starch, rice starch, and combinations thereof. In a eleventh example of the system, optionally including one or more or each of the first through tenth examples, the lignin is sourced from upcycled biomass feedstocks. In a twelfth example of the system, optionally including one or more or each of the first through eleventh examples, the nanoparticles are provided in a freeze-dried form configured for reconstitution before use. In a thirteenth example of the system, optionally including one or more or each of the first through twelfth examples, the composition is substantially free of alkali, and nanoparticle penetration into the cortex occurs primarily through nanoparticle-scale cuticle interstices and the coil-bend inflection (“Z-entry”) region, rather than chemically induced cuticle lifting. In a fourteenth example of the system, optionally including one or more or each of the first through thirteenth examples, the composition is configured to preserve cuticle integrity during treatment.

The disclosure also provides support for a method of treating hair comprising: applying to hair a composition comprising composite lignin-starch nanoparticles and a laccase enzyme at a pH of 5.5 to 6.0, permitting the nanoparticles to migrate through cuticle interstices into the cortex, and activating the laccase within the cortex to induce oxidative crosslink formation of carboxyl moieties of the lignin phase and establishment of a polymerized intrafiber scaffold physically distinct from keratin. In a first example of the method, nanoparticle migration includes entry through a coil-bend inflection region (“Z-entry junction”) exhibiting increased porosity due to angular torsion of the curl path. In a second example of the method, optionally including the first example, the polymerized scaffold forms a percolative network within interstitial spaces between keratin microfibrils.

The disclosure also provides support for a cosmetic hair treatment kit comprising: a first container including composite lignin-starch nanoparticles, a second container including a laccase enzyme in a cosmetically acceptable buffer, and instructions directing cortical migration followed by enzymatic activation to form an intrafiber scaffold. In a first example of the system, the system further comprises: an oxygen-permeable applicator configured to regulate oxygen exposure so that oxidative crosslink formation initiates within the cortex after nanoparticle penetration.

The present disclosure provides cosmetic compositions, methods, and treatment kits for structurally modifying keratinous fibers, particularly human hair, by forming a polymerized intrafiber scaffold within the cortex of the hair fiber. In contrast to conventional chemical straightening, relaxing, perming, or bond-repair treatments that rely on disulfide bond reduction, oxidation, or direct chemical modification of keratin proteins, the disclosed systems introduce a biodegradable nanoscale reinforcement matrix that is physically distinct from the keratin protein network of the hair fiber.

In one aspect, the invention provides hair treatment compositions comprising composite nanoparticles formed from a lignin phase and a starch phase, together with an oxidative enzyme system comprising laccase, dispersed in a cosmetically acceptable aqueous carrier having a mildly acidic pH compatible with scalp and hair cuticle integrity. The composite nanoparticles are dimensioned to migrate through cuticle interstices and localize within the cortex of the hair fiber. Following intrafiber localization, the laccase catalyzes oxidative coupling of phenolic moieties of the lignin phase, resulting in in situ polymerization and formation of a nanoscale intrafiber scaffold within the cortex. The scaffold is physically distinct from the native keratin protein network and reinforces the hair fiber without cleaving disulfide bonds of keratin.

In another aspect, the invention provides methods of treating hair that include applying the disclosed compositions to hair, allowing the composite lignin-starch nanoparticles to penetrate the cuticle and migrate into the cortex, and activating the laccase to catalyze polymerization of the lignin phase within the cortex to form the intrafiber scaffold. The scaffold formation provides mechanical reinforcement of keratin microfibrils and may modulate stress-strain behavior of the hair fiber, thereby improving properties such as tensile strength, elasticity, hydration retention, and resistance to breakage, while maintaining cuticle integrity and avoiding harsh alkaline conditions.

In further aspects, the invention provides cosmetic treatment kits including separate containers of the composite nanoparticles and the laccase enzyme, optionally in freeze-dried form for stability, together with instructions for reconstitution and application. The kits may optionally include pretreatment compositions, post-treatment sealants, shampoos, conditioners, and chromogenic formulations in which pigments are associated with or entrapped by the composite nanoparticles to provide color modification concurrently with structural reinforcement.

### BRIEF DESCRIPTION OF DRAWINGS

FIG. 1 is a schematic view illustrating composite lignin-starch nanoparticles and penetration of such nanoparticles through an intact cuticle layer into a cortex region of a hair fiber.

FIG. 2 is a schematic representation of enzyme-activated polymerization/enzyme-mediated polymerization of composite lignin-starch nanoparticles, illustrating laccase-mediated oxidative coupling and inter-nanoparticle association to initiate formation of a polymerized intrafiber scaffold.

FIG. 3 is a cross-sectional schematic view of a hair fiber showing formation of a polymerized intrafiber scaffold within the cortex region, the scaffold being physically distinct from the native keratin protein network, and exclusion of nanoparticles from a medulla region.

FIG. 4 illustrates a representative mechanical response of an untreated hair fiber under applied strain.

FIG. 5 illustrates a representative mechanical response of a treated hair fiber having an intrafiber nanoscale scaffold under applied strain, demonstrating increased tensile strength and elongation relative to untreated hair fibers.

FIG. 6 is a conceptual schematic representation of a nanoscale polymer network topology formed by polymerized lignin within the cortex (not to scale).

FIG. 7—Method Flow Diagram for Intrafiber Scaffold Formation
Table 1 illustrates representative example formulations of composite lignin-starch nanoparticle compositions suitable for intrafiber scaffold formation.
Table 2 illustrates representative two-part kit configurations and reconstitution parameters for forming an enzyme-activated intrafiber scaffold upon application to hair.
Table 3 illustrates representative manufacturing batch recipes and processing parameters for preparing composite lignin-starch nanoparticles.

DETAILED DESCRIPTION OF THE INVENTION

Examples of embodiments are provided so that this disclosure will be thorough and fully convey the scope to those skilled in the art. Numerous specific details are set forth, such as examples of specific components, devices, and methods, to provide a thorough understanding of the embodiments of the present disclosure. It will be apparent to those skilled in the art that specific details need not be employed, that example embodiments may be embodied in many different forms, and that neither should be construed to limit the scope of the disclosure. In some examples, embodiments, aspects, well-known processes, well-known device structures, and well-known technologies are not described in detail least one specification heading is required.

The terminology used herein is for the purpose of describing particular example embodiments only and is not intended to be limiting. As used herein, the singular forms “a,” “an,” and “the” may be intended to include the plural forms as well unless the context clearly indicates otherwise. The terms “comprises,” “comprising,” “including,” and “having,” are inclusive and therefore specify the presence of stated features, integers, steps, operations, elements, and/or components but do not preclude the presence or addition of one or more other features, integers, steps, operations, elements, components, and/or groups thereof. The method steps, processes, and operations described herein are not to be construed as necessarily requiring their performance in the particular order discussed or illustrated unless specifically identified as an order of performance. It is also to be understood that additional or alternative steps may be employed.

The preceding summary, as well as the following detailed description of certain embodiments, will be better understood when read in conjunction with the appended figure of experimental data and results. As used herein, an element or step recited in the singular and proceeded with the word “a” or “an” should be understood as not excluding the plural of said elements or steps unless such exclusion is explicitly stated. Furthermore, references to “one embodiment” or “an embodiment” are not intended to be interpreted as excluding the existence of additional embodiments that also incorporate the recited features. Moreover, unless explicitly stated to the contrary, embodiments “comprising” or “having” an element or a plurality of elements having a particular property may include additional such elements not having that property. When a definition is provided herein, it supersedes any other meaning or definition.

Definitions. For clarity and to avoid ambiguity, the following terms used in this disclosure are defined as follows:
“Composite lignin-starch nanoparticles.”

Refers to biodegradable particulate structures comprising a lignin phase and/or a starch phase within a single particle, including, but not limited to, heterogeneous domain distributions and/or core-shell, interpenetrating, and/or phase-separated morphologies, and/or any combination thereof.
“Lignin phase.”

Refers to lignin-derived aromatic polymer domains, including, but not limited to, lignin obtained from plant biomass by organosolv, kraft, soda, and/or other extraction processes, and/or any combination thereof.
“Starch phase.”

Refers to polysaccharide domains derived from starch sources, including, but not limited to, corn, potato, tapioca, rice, and/or modified starches, and/or any combination thereof.
“Nanoparticle.”

Refers to a particle having at least one characteristic dimension in the nanometer scale, including, but not limited to, particles having an average diameter in a range of about 1 nm to about 5,000 nm, and/or any subrange thereof, and/or particles having non-spherical, irregular, aggregated, and/or anisotropic forms, and/or any combination thereof.

As used herein, “nanostructures” and “nanoparticles” encompass composite lignin-starch particulate and clustered structures capable of forming intrafiber scaffolds, and are not limited to any single morphology.

“As used herein, ‘composite nanoparticles’refer to nanocomposite particles comprising at least two distinct material phases, including a lignin phase and a starch phase, within a single nanoscale particle.
“Average diameter.”

Refers to number-average, volume-average, and/or intensity-average particle diameter as determined by dynamic light scattering (DLS), nanoparticle tracking analysis (NTA), electron microscopy, and/or other suitable particle sizing techniques, and/or any combination thereof.
“Intrafiber scaffold.”

Refers to a polymerized network, matrix, and/or interconnected structure formed within the interior of a hair fiber, particularly within the cortex, that is physically distinct from the native keratin protein network of the hair fiber and provides internal mechanical reinforcement, the scaffold being formed in situ by polymerization, aggregation, association, and/or any combination thereof.
“Physically distinct from keratin.”

Means that the scaffold constitutes a separate polymeric phase from keratin microfibrils and does not consist of covalent modification and/or direct crosslinking of keratin protein chains, while not requiring complete spatial separation and permitting partial interpenetration and/or interfacial contact, provided that the scaffold constitutes a separate polymeric phase.
“Keratin microfibrils.”

Refers to filamentous keratin protein assemblies within the cortex of the hair fiber, including, but not limited to, intermediate filament bundles and/or associated matrix components, and/or any combination thereof.
“Cuticle interstices.”

Refers to gaps, channels, and/or pathways between overlapping cuticle scales through which nanoparticles may migrate into the cortex under cosmetic treatment conditions.

As used herein, “interstitial spaces” refers to micro-scale voids, channels, or regions between adjacent keratin microfibrils within the cortex that permit localization and distribution of polymeric material without covalent modification of keratin.
“Phenol-oxidizing enzyme.”

Refers to an enzyme capable of catalyzing oxidative coupling of phenolic moieties, including, but not limited to, laccases, peroxidases, tyrosinases, and/or related oxidoreductases, and/or any combination thereof.
“Phenol-oxidizing enzymes.”

Includes direct oxidases and/or enzyme-mediator systems capable of catalyzing oxidative coupling of lignin phenolic moieties, and/or any combination thereof.
“Laccase.”

Refers to a multicopper oxidase enzyme capable of catalyzing oxidation of phenolic substrates, including, but not limited to, lignin-derived phenolic moieties, under cosmetic treatment conditions.
“Cosmetically acceptable carrier.”

Refers to a carrier and/or vehicle suitable for topical application to hair and/or scalp, including, but not limited to, aqueous, aqueous-based, emulsified, gelled, foamed, sprayable, and/or encapsulated formulations, and/or any combination thereof, compatible with cosmetic safety standards.
“Substantially free of alkali sufficient to cause cuticle lifting.”

Refers to formulations that do not contain strongly alkaline components in amounts that would raise pH to levels known to cause cuticle swelling and/or lifting under typical hair treatment conditions.

The term “fullerene-inspired” as used herein refers to geometric arrangement or network topology rather than chemical composition, and encompasses biodegradable polymeric nanostructures exhibiting closed, semi-closed, or multi-domain morphologies suitable for cortical penetration and intrafiber scaffold formation.

The disclosed systems are based on the recognition that internal reinforcement of hair fibers can be achieved by introducing a biodegradable polymeric scaffold into the cortex of the hair fiber, rather than by chemically modifying keratin proteins or disulfide bonds. To this end, composite nanoparticles comprising a lignin phase and/or a starch phase are provided as a delivery vehicle and/or structural precursor. Lignin provides phenolic moieties capable of oxidative coupling and/or polymerization, while starch contributes biocompatibility, dispersibility, and/or controlled nanoparticle formation. The composite particles are biodegradable and plant-derived, and may be formed using lignin selected from organosolv lignin, kraft lignin, or any combination thereof, and starch selected from corn starch, potato starch, tapioca starch, or any combination thereof.

In certain embodiments, the composite lignin-starch nanoparticles may adopt generally spherical, quasi-spherical, polyhedral, or multi-faceted geometries. Such geometries may exhibit overall symmetry or topology conceptually analogous to fullerene-like or cage-like architectures, without comprising carbon allotropes, graphene derivatives, or true fullerene materials.

The composite nanoparticles are formulated with a phenol oxidase enzyme, such as laccase enzyme in a cosmetically acceptable aqueous carrier maintained at a mildly acidic pH, for example, about 5.5 to about 6.0.; furthermore, for example, pH of 5, 5.1, 5.2, 5.3, 5.4, 5.5, 5.6, 5.7, 5.8, 5.9, 6.0, 6.1, 6.2, 6.3, 6.4, and/or 6.5. This pH range is compatible with scalp physiology and cuticle integrity and avoids cuticle lifting or damage associated with strongly alkaline hair treatments. The nanoparticle size distribution is selected to facilitate migration through cuticle interstices into the cortex of the hair fiber. Once localized within the cortex, the phenol oxidase enzyme, such as laccase, catalyzes oxidative coupling of phenolic groups of the lignin phase, causing in situ polymerization of lignin and/or formation of a nanoscale intrafiber scaffold.

In certain embodiments, oxidative crosslink formation involves phenolic moieties of lignin and may further involve carboxyl-functionalized lignin groups that participate in radical-mediated coupling reactions and/or mixed ether or ester linkages.

The disclosed technology relates to biodegradable, cosmetically compatible hair treatment systems based on composite lignin-starch nanoparticles configured for delivery into the cortex of keratin fibers. Following intrafiber localization, an enzyme system (e.g., laccase) catalyzes oxidative coupling of lignin phenolic moieties to form a three-dimensional intrafiber scaffold that reinforces the internal architecture of the hair fiber without chemical modification of keratin disulfide bonds.

In contrast to bond builders, straightening systems, and surface film-formers, the disclosed compositions achieve internal mechanical reinforcement through enzyme-activated polymerization/enzyme-mediated polymerization within the cortex under non-caustic pH conditions; example, pH of 5, 5.1, 5.2, 5.3, 5.4, 5.5, 5.6, 5.7, 5.8, 5.9, 6.0, 6.1, 6.2, 6.3, 6.4, and/or 6.5. and cosmetically acceptable temperature conditions. The resulting scaffold distributes mechanical stress, improves tensile strength and elasticity, and enhances resistance to humidity-induced deformation while preserving natural movement and texture.

In certain embodiments, optical stabilizers or chroma-mitigating additives may be included to maintain cosmetic transparency and color safety during enzyme activation. The compositions, methods, kits, and manufacturing processes described herein provide a non-toxic, biodegradable platform for intrafiber structural reinforcement of keratinous fibers, distinct from chemical relaxers, aldehyde-based treatments, peptide repair systems, and conventional conditioning polymers.

In certain embodiments, the composite lignin-starch particles and/or polymerized domains formed therefrom may adopt generally spherical and/or quasi-spherical nanostructures that are capable of self-organization within the cortex during enzyme-triggered oxidative polymerization. Such nanostructures may associate to form localized reinforced regions and/or lattice-like arrangements within the intrafiber scaffold.

In certain embodiments, the nanostructures are generally spherical, quasi-spherical, and/or polyhedral.

In some embodiments, formation of the intrafiber scaffold may preferentially occur and/or provide enhanced mechanical reinforcement at regions of higher local curvature and/or mechanical stress within the hair fiber, such as along intrinsic curl paths and/or localized curvature maxima. Without being bound by theory, such localized reinforcement may reduce fracture and/or fatigue at curvature vertices and improve flexibility, strength, and moisture retention of curved or coiled hair geometries.

In certain embodiments, the nanostructures or scaffold domains may exhibit heterogeneous internal and external regions, including, but not limited to, relatively hydrophobic exterior domains configured to reduce humidity-induced deformation and frizz, and relatively hydrophilic interior domains configured to buffer internal moisture within the cortex.

In certain embodiments, the intrafiber scaffold preferentially reinforces regions of higher curvature or localized stress concentration along the hair fiber, thereby stabilizing natural curl geometry and reducing fracture at curvature vertices.

In some embodiments, enzyme activity and/or polymerization kinetics may be modulated by controlling oxygen exposure, buffering systems, oxygen scavengers, and/or formulation additives to avoid over-polymerization and/or undesirable chromogenic reactions associated with lignin oxidation. In certain embodiments, additives and/or post-treatments may be employed to neutralize or mitigate undesired color changes associated with enzymatic polymerization while preserving cosmetic transparency or color neutrality.

In certain embodiments, chroma-mitigating or color-neutralizing agents may be included in the formulation to reduce or mask oxidative chromogenic byproducts associated with enzyme-mediated polymerization of lignin. Non-limiting examples include chlorophyll derivatives, chlorophyllin complexes, plant-derived porphyrins, carotenoid analogs, optical brighteners, and cosmetically acceptable color-correcting agents. Such additives may be selected to maintain cosmetic transparency or color safety of treated hair while remaining compatible with enzyme activity and nanoparticle stability.

In further embodiments, the composite lignin-starch particles may be processed and/or formulated to produce generally spherical nanostructures having diameters in a nanoscale range suitable for penetration into the cortex, for example, about 30 nm to about 200 nm, and may be stabilized by freeze-drying, microencapsulation, and/or other cosmetic-compatible stabilization techniques. For example, 30, 35, 30, 35, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, etc., and/or any fraction thereof.

In various embodiments, additional cosmetic excipients, including conditioning agents, botanical extracts, oils, silicones, fragrances, polymers, or combinations thereof, may be included, provided that such excipients do not substantially interfere with the formation, stability, or function of the nanostructures or intrafiber scaffold.

In certain embodiments, the composite nanoparticles are sized to migrate through cuticle interstices having characteristic dimensions on the order of tens to hundreds of nanometers. For example, in some embodiments, the cuticle interstices have characteristic dimensions in a range of about 50 nm to about 200 nm, and the composite nanoparticles are sized accordingly to facilitate migration into the cortex, although recognizing that interstice dimensions may vary with hair type, condition, and hydration state. For example, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, 150 nm, 155 nm, 160 nm, 165 nm, 170 nm, 175 nm, 180 nm, 185 nm, 190 nm, 195 nm, 200 nm, etc., and/or any fraction thereof.

The resulting intrafiber scaffold is physically distinct from the native keratin protein network of the hair fiber. Rather than repairing and/or reforming disulfide bonds, the scaffold forms an internal reinforcement matrix that occupies interstitial spaces around keratin microfibrils, thereby mechanically reinforcing the hair fiber. Because scaffold formation does not require cleavage of keratin disulfide bonds, the treatment avoids the irreversible chemical damage associated with traditional relaxing, perming, and/or straightening chemistries. The scaffold may be configured to improve tensile properties, elasticity, hydration retention, and resistance to breakage, and may provide controlled modulation of curl pattern or shape retention through mechanical reinforcement rather than protein crosslinking. For example, in certain embodiments, the treated hair fibers exhibit an increase in tensile strength of about 5% to about 28% relative to untreated control hair fibers, as measured by standardized single-fiber tensile testing under comparable humidity and temperature conditions. For example, in certain embodiments, the treated hair fibers exhibit an increase in tensile strength of about 5% to about 28% relative to untreated control hair fibers, and in some embodiments relative to hair fibers treated with conventional conditioning compositions. For example, in certain embodiments, the treated hair fibers are expected to exhibit an increase in tensile strength of about 5% to about 28% relative to untreated control hair fibers, when evaluated using standard mechanical testing protocols. For example, the increase in the tensile strength in % of about 5, 6, 7, 8, 9, 10, 11, 12, 13, 14,1 15, 16, 17, 18, 19, 20, 21, 22, 23, 24, 25, 26, 27, 28%, etc., and any fraction thereof.

In certain embodiments, localization of the composite nanoparticles and formation of the intrafiber scaffold occur predominantly within the cortex and are substantially excluded from the medulla of the hair fiber.

In certain embodiments, the composite nanoparticles and enzyme system are provided in a multi-component kit. The nanoparticles may be provided in freeze-dried form in a first container, with a phenol oxidase enzyme, such as laccase enzyme, provided in a second container, allowing reconstitution shortly before use to enhance shelf stability. The kit may further include a pretreatment composition to prepare the hair for nanoparticle penetration and/or a post-treatment sealant to condition and/or protect the hair following scaffold formation. In chromogenic embodiments, pigments may be associated with and/or entrapped by the composite nanoparticles, enabling simultaneous color deposition and structural reinforcement within the cortex.

The disclosed compositions, methods, and kits provide a non-caustic, biomaterial-based approach to hair modification that fundamentally differs from prior approaches that rely on keratin crosslinking, disulfide bond chemistry, reducing sugars, peptide repair systems, and/or generic in situ crosslinking networks. By introducing a polymerized intrafiber scaffold composed of biodegradable lignin-starch nanoparticles, the invention enables internal structural reinforcement of hair fibers while preserving the native keratin architecture and cuticular integrity.

In certain embodiments, the disclosed compositions and methods are formulated without reactive aldehydes, formaldehyde donors, glyoxal systems, high-pH relaxer chemistries, peroxide-based oxidants, graphene derivatives, or cytotoxic penetration enhancers. The system operates under cosmetically acceptable pH and temperature conditions and relies on biodegradable polymer phases and enzyme-mediated crosslink formation rather than harsh chemical swelling, denaturation, or keratin bond disruption.

In certain embodiments, the composite nanoparticles may comprise different relative weight ratios of the lignin phase to the starch phase to tailor penetration behavior, polymerization kinetics, and scaffold density within the cortex. For example, higher lignin content may be selected to increase scaffold rigidity, while higher starch content may be selected to increase dispersibility and flexibility of the scaffold. In some embodiments, the lignin-to-starch weight ratio may be, but not limited to, in a range of about 20:80 to about 80:20. In certain embodiments, the lignin-to-starch weight ratio may be about 30:70, about 40:60, about 50:50, about 60:40, or about 70:30. In certain embodiments, ratios outside these ranges may be employed depending on hair type, treatment objectives, and formulation constraints.

In certain embodiments, the lignin phase comprises chemically functionalized lignin derivatives, including carboxyl-functionalized lignin, esterified lignin, oxidized lignin, sulfonated lignin, or combinations thereof. Carboxyl-functionalized lignin may include lignin that has been selectively oxidized, grafted, or derivatized to introduce carboxylic acid and/or carboxylate groups along the lignin backbone and/or side chains. Such functionalization may increase aqueous dispersibility, modulate nanoparticle surface charge, and provide additional reactive sites for enzyme-mediated crosslink formation within the cortex following intrafiber localization of the composite nanoparticles.

In some embodiments, the lignin phase may be derived from different plant sources and/or extraction processes, including organosolv lignin, kraft lignin, soda lignin, and/or any combination thereof. The starch phase may be derived from different botanical sources, including corn starch, potato starch, tapioca starch, rice starch, and/or modified starches, to tune nanoparticle stability and/or interactions with hair fibers.

In further embodiments, the composite nanoparticles may be configured with alternative internal morphologies, including, but not limited to, core-shell configurations in which one phase is enriched at a surface region of the nanoparticle, and/or heterogeneous domain distributions in which lignin-rich and/or starch-rich domains are interspersed within a single nanoparticle. Such variations may be used to modulate surface charge, hydrophilicity, and/or enzyme accessibility.

In certain embodiments, the composite nanoparticles are formulated to exhibit a surface zeta potential selected to promote electrostatic interaction with keratinous fibers and/or facilitate intrafiber localization within the cortex.

In certain embodiments, a phenol oxidase enzyme, such as laccase enzyme is employed to catalyze oxidative polymerization of lignin phenolic moieties; in other embodiments, alternative oxidative enzyme systems may be employed, including, but not limited to, phenol oxidases, peroxidases, and/or any combination thereof, provided that such systems catalyze polymerization of lignin-derived moieties within the cortex.

In some embodiments, enzyme activation may be modulated by temperature, oxygen availability, and/or formulation additives that control reaction kinetics. For example, polymerization may be initiated and/or accelerated under ambient oxygen conditions and/or mild thermal activation compatible with cosmetic use. In certain embodiments, enzyme inhibitors and/or stabilizers may be included to delay polymerization until after the nanoparticles have localized within the cortex.

In one embodiment, a phenol oxidase enzyme, such as lactase, catalyzes the oxidative coupling of phenolic moieties in the lignin phase after intrafiber localization of the composite nanoparticles.

In certain embodiments, other phenol-oxidizing enzymes capable of catalyzing the oxidative coupling of lignin phenolic moieties may be used in place of, and/or in combination with, a phenol oxidase enzyme, such as laccase. For example, suitable enzymes may include, but are not limited to, peroxidases (including, but not limited to, horseradish peroxidase, lignin peroxidase, and/or manganese peroxidase), phenol oxidases, tyrosinases, and/or other oxidoreductases capable of catalyzing phenolic coupling reactions under cosmetically acceptable conditions. In certain embodiments, enzyme systems may further include redox mediators to facilitate oxidation of lignin phenolic moieties.

In certain embodiments, lignin-starch nanoparticles are configured to penetrate the hair cuticle and localize within the cortex, where enzyme-activated polymerization/enzyme-mediated polymerization induces formation of an intrafiber scaffold that reinforces the hair structure without penetrating the medulla.

In certain embodiments, oxidative crosslink formation within the intrafiber scaffold involves phenolic moieties of lignin, carboxyl-functionalized lignin moieties, and/or combinations thereof. In addition to phenolic radical coupling, enzyme-mediated oxidative activation may promote formation of covalent linkages involving carboxyl-containing lignin moieties, including C—C, C—O—C, and mixed ester- or ether-type linkages formed through radical-mediated reactions and/or mediator-assisted coupling mechanisms. Such crosslink formation contributes to fusion of adjacent composite nanoparticles into a continuous three-dimensional intrafiber scaffold within the cortex.

In various embodiments, the composite nanoparticles and/or enzyme system may be provided in alternative cosmetic formulation formats, including creams, lotions, gels, foams, sprays, serums, emulsions, and/or encapsulated delivery systems. Such formats may be selected based on desired application feel, spreadability, residence time on hair fibers, and/or compatibility with existing hair care routines.

In some embodiments, the nanoparticles and/or enzyme may be co-formulated in a single composition, while in other embodiments, they may be provided in separate compositions applied sequentially to the hair to provide greater control over penetration and polymerization timing.

In further embodiments, the nanoparticles may be provided in dry, freeze-dried, and/or rehydratable powder form for improved shelf stability and activated upon reconstitution prior to application.

In certain embodiments, the method of treatment may involve a single-step application of the composition to wet or dry hair, while in other embodiments the method may include multi-step protocols involving pretreatment to cleanse and/or prime the hair fiber, followed by application of the scaffold-forming composition and optional post-treatment conditioning and/or sealing steps.

In some embodiments, the treatment may be configured as a one-time application providing semi-permanent structural reinforcement, whereas in other embodiments, repeated and/or periodic applications may be employed to maintain or progressively build scaffold density within the cortex over time.

In certain embodiments, the composite lignin-starch nanostructures self-assemble into particulate or clustered morphologies exhibiting heterogeneous internal and external domain distributions. For example, in some embodiments, relatively hydrophobic lignin-enriched domains may be preferentially expressed at or near exterior surfaces of the nanostructures, while relatively hydrophilic starch-enriched domains may be preferentially distributed within interior regions, or vice versa, depending on formulation conditions, lignin fractionation, surface modification, or processing history. Such heterogeneous domain arrangements may facilitate controlled interaction with the hair fiber environment, including reduced surface wetting resistance, modulation of humidity sensitivity, internal moisture buffering within the cortex, and improved compatibility with aqueous cosmetic carriers. The term “hydrophobic exterior domains” and “hydrophilic internal compartments” encompasses any such relative domain enrichment and is not limited to any particular geometric configuration.

In further embodiments, treatment parameters, such as contact time, temperature, and/or moisture level, may be adjusted to tune scaffold formation kinetics and the resulting mechanical properties of the hair fibers.

In various embodiments, the intrafiber scaffold may be configured primarily to increase tensile strength and reduce breakage of hair fibers. In other embodiments, the scaffold may be configured to modulate flexibility, retention of curl pattern, and/or resistance to humidity-induced shape changes by adjusting scaffold density and distribution within the cortex.

In some embodiments, the scaffold-forming treatment may be combined with chromogenic and/or color-delivering systems in which pigments and/or dyes are associated with the composite nanoparticles, enabling simultaneous color modification and structural reinforcement. In other embodiments, the treatment may be configured as a neutral, color-safe strengthening and/or conditioning treatment.

In further embodiments, the compositions and methods described herein may be applied to keratinous fibers of different types, including human scalp hair, facial hair, animal fur, wool fibers, and/or other keratin-based fibers, with formulation parameters adjusted to accommodate differences in fiber diameter, cuticle structure, and/or porosity.

In certain embodiments, the cosmetic treatment kit may include separate containers for the composite nanoparticles, enzyme system, pretreatment compositions, and/or post-treatment sealants. In other embodiments, the kit may include applicators, mixing vessels, and/or single-use packaging formats to facilitate controlled preparation and application of the scaffold-forming composition.

In certain embodiments, the kit comprises multi-chamber packaging configured to separate the composite nanoparticles and/or enzyme system prior to use, and/or applicators configured to regulate oxygen exposure during application to control enzyme activation and polymerization timing.

In some embodiments, the kit may be configured for professional salon use, with larger-volume containers and extended activation times, while in other embodiments, the kit may be configured for consumer home use with simplified instructions and pre-measured components.

In certain embodiments, the methods of use described herein may be performed in different orders than those expressly described, and one or more steps may be added, omitted, repeated, or combined without departing from the scope of the invention. For example, pretreatment, penetration, enzyme activation, polymerization, rinsing, conditioning, or sealing steps may be performed in alternative sequences, in parallel, or in staged cycles depending on formulation format, hair type, treatment objectives, and application context. In certain embodiments, one or more optional steps may be omitted, and in other embodiments, additional steps may be included, provided that formation of the intrafiber scaffold within the cortex is achieved.

In various alternative embodiments, the composition, method, and kit described herein may be implemented with variations in nanoparticle composition, enzyme systems, formulation formats, treatment protocols, and functional objectives, without departing from the scope of the invention.

The systems, compositions, and methods described herein provide a platform for intrafiber scaffold formation within keratinous fibers. In certain embodiments and extensions, the core concepts disclosed herein may be adapted, expanded, and/or combined with additional technologies to enable new functionalities, product formats, and application domains.

In certain embodiments, the composite nanoparticles comprise lignin in an amount of about 1-99 wt %, 5-95 wt %, 10-90 wt %, 20-80 wt %, 25-75 wt %, 30-70 wt %, 35-65 wt %, 40-60 wt %, 45-55 wt %, 50-50 wt %, 55-45 wt %, and/or 60-40 wt %, and/or starch in complementary amounts of about 99-1 wt %, 95-5 wt %, 90-10 wt %, 80-20 wt %, 75-25 wt %, 70-30 wt %, 65-35 wt %, 60-40 wt %, 55-45 wt %, 50-50 wt %, 45-55 wt %, and/or 40-60 wt % (based on solids in the nanoparticle blend), including, but not limited to, any subrange and/or ratio and/or combination thereof. Such ratios may be selected and/or adjusted to balance scaffold rigidity, dispersibility, penetration behavior, polymerization kinetics, cosmetic transparency, and/or enzyme accessibility. In certain embodiments, lignin fractions may be selected, modified, fractionated, and/or processed to tune phenolic content, molecular weight distribution, and/or reactivity to balance polymerization kinetics with optical clarity and/or color stability.

In certain embodiments, the composite nanoparticles exhibit average diameters in a range of about 1 nm to about 5,000 nm, 10 nm to about 2,000 nm, 20 nm to about 1,000 nm, 30 nm to about 800 nm, 40 nm to about 600 nm, 50 nm to about 500 nm, 60 nm to about 400 nm, 70 nm to about 300 nm, 80 nm to about 250 nm, 90 nm to about 200 nm, 95 nm to about 180 nm, 98 nm to about 160 nm, 100 nm to about 150 nm, 110 nm to about 140 nm, 120 nm to about 130 nm, and/or any subrange and/or discrete value encompassed therein. In certain embodiments, the nanoparticles exhibit a polydispersity index (PDI) in a range of about 0.01 to about 0.9, 0.05 to about 0.7, 0.1 to about 0.6, 0.15 to about 0.5, 0.2 to about 0.4, and/or any subrange and/or discrete value encompassed therein. In certain embodiments, the nanoparticles exhibit a zeta potential in a range of about −1 mV to about 100 mV, −5 mV to about −80 mV, −10 mV to about −60 mV, −15 mV to about −50 mV, 18 mV to about −42 mV, and/or any subrange and/or discrete value encompassed therein, which may reduce agglomeration and/or improve colloidal stability prior to activation. Nanoparticle size, size distribution, and/or surface properties may be characterized by dynamic light scattering (DLS), nanoparticle tracking analysis (NTA), electrophoretic mobility measurements, electron microscopy, and/or any other suitable particle characterization technique, and/or any combination thereof.

In certain embodiments, the enzyme system comprises a phenol oxidase enzyme, such as laccase present at an activity of about 0.01-500 U/mL, 0.1-200 U/mL, 0.5-100 U/mL, 1-50 U/mL, 2-40 U/mL, 5-30 U/mL, 10-25 U/mL, 15-20 U/mL, and/or any subrange and/or discrete value encompassed therein, and at a formulation loading of about 0.001-10 wt %, 0.005-5 wt %, 0.01-2.0 wt %, 0.02-1.0 wt %, 0.05-0.8 wt %, 0.1-0.5 wt %, and/or any subrange and/or discrete value encompassed therein. Polymerization may be activated under ambient oxygen and/or controlled oxygen conditions at temperatures of about 5° C. to about 70° C., 10° C. to about 60° C., 15° C. to about 55° C., 20° C. to about 45° C., 25° C. to about 40° C., 29° C. to about 35° C., and/or any subrange and/or discrete value encompassed therein. In certain embodiments, formulation conditions are selected and/or adjusted to preserve enzyme stability during storage and/or transport and to permit controlled activation during use and/or application.

In certain embodiments, formulations may be maintained at a pH in a range of about 4.0-8.5, 4.5-8.0, 5.0-7.5, 5.2-6.8, 5.5-6.5, 5.5-6.0, and/or any subrange and/or discrete value encompassed therein during manufacturing, storage, reconstitution, and/or use, and rheological properties may be adjusted to promote uniform deposition and adequate residence time for penetration, including, but not limited to, pre-activation viscosities of about 10 cP to about 20,000 cP, 50 cP to about 10,000 cP, 100 cP to about 6,000 cP, 300 cP to about 4,000 cP, 500 cP to about 2,500 cP, 800 cP to about 1,500 cP, and/or any subrange and/or discrete value encompassed therein.

In certain embodiments, composite nanoparticles are stabilized by freeze-drying, spray-drying, vacuum-drying, and/or other dehydration techniques, achieving residual moisture contents of about 0.1-15 wt %, 0.5-10 wt %, 1-8 wt %, 1-5 wt %, 1-3 wt %, and/or any subrange and/or discrete value encompassed therein, and glass transition temperatures of about 20° C. to about 250° C., 50° C. to about 220° C., 80° C. to about 200° C., 100° C. to about 180° C., 110° C. to about 180° C., and/or any subrange and/or discrete value encompassed therein, facilitating rehydration with reduced variability. In certain embodiments, rehydration variability of average particle size is within about ±1% to about ±50%, ±2% to about ±30%, ±5% to about ±20%, ±10% to about ±15%, and/or any subrange and/or discrete value encompassed therein relative to pre-drying values.

In certain embodiments, nanoparticle localization within the hair fiber occurs predominantly within the cortex to depths of about 0.1 μm to about 100 μm, 0.5 μm to about 50 μm, 1 μm to about 30 μm, 2 μm to about 25 μm, 3 μm to about 20 μm, 4 μm to about 18 μm, 5 μm to about 15 μm, 6 μm to about 12 μm, and/or any subrange and/or discrete value encompassed therein, measured from an outer cuticle surface, while remaining substantially excluded from the medulla by at least about 0.1 μm to about 20μm, 0.5 μm to about 10 μm, 1 μm to about 5 μm, 2 μm to about 4 μm, and/or any subrange and/or discrete value encompassed therein. Intrafiber localization, penetration depth, scaffold formation, and/or spatial distribution may be assessed by scanning electron microscopy (SEM), transmission electron microscopy (TEM), confocal microscopy, fluorescence labeling, X-ray microtomography, and/or any other suitable imaging technique, and/or any combination thereof. Changes in mechanical properties may be assessed by atomic force microscopy (AFM), nanoindentation, micro-tensile testing, and/or other suitable micromechanical methods, and polymerization signatures of lignin and/or scaffold formation may be assessed by Raman spectroscopy, Fourier transform infrared (FTIR) spectroscopy, solid-state NMR, and/or other suitable spectroscopic techniques, and/or any combination thereof. Mechanical performance may be further evaluated by single-fiber tensile testing, dynamic mechanical analysis (DMA), cyclic fatigue testing, and/or environmental mechanical testing under varying humidity, temperature, and/or moisture conditions.

In certain embodiments, formulation excipients including conditioning agents, botanical extracts, oils, silicones, fragrances, polymers, and/or humectants may be included, but not limited to, provided such excipients do not substantially interfere with nanoparticle integrity, penetration, and/or enzyme activity. In some embodiments, humectant systems (e.g., glycerin- and/or aloe-based aqueous media) may be employed; in other embodiments, such components may be excluded based on product objectives and stability considerations.

In certain embodiments, scaffold reversibility or remodeling may be achieved by applying a terpenoid-containing solvent system (e.g., limonene in a cosmetic oil carrier) configured to disrupt non-covalent interactions within the polymerized lignin-starch network without cleaving keratin disulfide bonds.

In certain embodiments, the composite lignin-starch nanoparticles and enzyme system are delivered using aqueous, hydroalcoholic, and/or nano-emulsion-based vehicles configured to promote uniform wetting of the cuticle and controlled delivery into the cortex, including, but not limited to, oil-in-water emulsions, water-in-oil emulsions, bicontinuous emulsions, microemulsions, and/or any combination thereof. In certain embodiments, nano-emulsion droplets have an average diameter of about 5 nm to about 1,000 nm, 10 nm to about 800 nm, 20 nm to about 500 nm, 40 nm to about 300 nm, 60 nm to about 250 nm, 80 nm to about 200 nm, 100 nm to about 180 nm, and/or any subrange and/or discrete value encompassed therein. In certain embodiments, the formulation is adjusted to a pre-activation viscosity of about 1 cP to about 50,000 cP, 10 cP to about 20,000 cP, 50 cP to about 10,000 cP, 100 cP to about 6,000 cP, 300 cP to about 4,000 cP, 500 cP to about 2,500 cP, 800 cP to about 1,500 cP, and/or any subrange and/or discrete value encompassed therein to balance spreadability, wetting, film formation, and/or residence time on hair fibers.

In certain embodiments, the formulation includes buffering agents configured to maintain cosmetically compatible pH during penetration, enzyme stabilization, enzyme activation, and/or scaffold formation, including, but not limited to, citrate buffers, acetate buffers, phosphate buffers, lactate buffers, succinate buffers, histidine buffers, Good's buffers, and/or any combination thereof. Buffer concentrations may be selected in a range of about 1 μM to about 500 mM, 5 μM to about 200 mM, 10 μM to about 100 mM, 50 μM to about 50 mM, 100 μM to about 10 mM, 0.5 mM to about 25 mM, 1 mM to about 20 mM, and/or any subrange and/or discrete value encompassed therein to maintain formulation pH in a range of about 4.0 to about 8.5, 4.5 to about 7.5, 5.0 to about 6.8, 5.2 to about 6.5, 5.5 to about 6.2, 5.5 to about 6.0, and/or any subrange and/or discrete value encompassed therein during use.

In certain embodiments, hair-safe penetration facilitators, wetting agents, dispersants, and/or deposition aids may be included at low levels to improve wetting, transport, and/or intrafiber delivery without chemically modifying keratin disulfide bonds, including, but not limited to, non-ionic surfactants, amphoteric surfactants, polymeric dispersants, lightweight plant-derived esters, fatty alcohols, glyceryl esters, silicones, volatile carriers, and/or any combination thereof. In certain embodiments, such components are present at levels of about 0.001-10 wt %, 0.01-5 wt %, 0.05-3 wt %, 0.1-3 wt %, 0.1-2 wt %, and/or any subrange and/or discrete value encompassed therein, and aqueous dispersion media may be formulated with controlled ionic strength of about 0.01 mM to about 500 mM, 0.1 mM to about 200 mM, 1 mM to about 100 mM, 1 mM to about 50 mM, and/or any subrange and/or discrete value encompassed therein. In certain embodiments, harsh penetration enhancers, strongly alkaline agents, oxidative bleaches, reducing agents, and/or disulfide-cleaving chemistries are excluded.

In certain embodiments, nanoparticle size is selected and/or adjusted to correlate with penetration depth, residence time, and/or spatial distribution within the cortex of a hair fiber. By way of non-limiting example, composite nanoparticles having average diameters of about 1 nm to about 5,000 nm, 10 nm to about 2,000 nm, 20 nm to about 1,000 nm, 40 nm to about 600 nm, 60 nm to about 400 nm, 80 nm to about 250 nm, 90 nm to about 200 nm, 95 nm to about 180 nm, 98 nm to about 160 nm, 100 nm to about 150 nm, 110 nm to about 140 nm, 120 nm to about 130 nm, and/or any subrange and/or discrete value encompassed therein may achieve penetration depths of about 0.1 μm to about 100μm, 0.5 μm to about 50 μm, 1 μm to about 30 μm, 2 μm to about 25 μm, 3 μm to about 20 μm, 4 μm to about 18 μm, 5 μm to about 15 μm, and/or any subrange and/or discrete value encompassed therein, measured from an outer cuticle surface, while remaining substantially excluded from the medulla by at least about 0.1 μm to about 20 μm, 0.5 μm to about 10 μm, 1 μm to about 5 μm, 2 μm to about 4 μm, and/or any subrange and/or discrete value encompassed therein. In certain embodiments, penetration depth, localization, and/or size-dependent transport behavior may be tuned by formulation parameters, application time, moisture content, temperature, cuticle condition, ionic strength, and/or any combination thereof.

In certain embodiments, the intrafiber scaffold provides mechanical reinforcement and curl stabilization without deposition of chromogenic species, dyes, pigments, or melanin-interacting compounds, such that the treatment is color-neutral and color-safe.

In certain embodiments, the method proceeds through a staged and/or continuous sequence comprising (i) delivery, deposition, wetting, and/or surface conditioning of the formulation on the hair fiber, (ii) penetration and/or diffusion of composite nanoparticles through cuticle interstices into the cortex, and (iii) enzyme activation and/or scaffold formation within the cortex under ambient oxygen, controlled oxygen, and/or cosmetically acceptable thermal conditions, including temperatures of about 5° C. to about 80° C., 10° C. to about 70° C., 15° C. to about 60° C., 20° C. to about 55° C., 25° C. to about 50° C., 29° C. to about 45° C., and/or any subrange and/or discrete value encompassed therein. In certain embodiments, penetration and/or intrafiber delivery is facilitated by mild mechanical handling, including, but not limited to, combing, brushing, massaging, vibration, and/or other low-shear mechanical actions, and/or by transient increases in cuticle lamellar spacing induced by mild thermal exposure, hydration, osmotic effects, ionic strength modulation, and/or surfactant-mediated wetting, without inducing chemical swelling, oxidative damage, and/or cuticle lifting.

In certain embodiments, the lignin-starch intrafiber scaffold modifies and/or enhances the mechanical behavior of the hair fiber by reinforcing the internal architecture of the cortex. Rather than breaking, rearranging, and/or chemically modifying disulfide bonds, the systems and methods described herein introduce a biodegradable structural network that distributes mechanical stress, stabilizes curl geometry and/or fiber curvature, and improves fiber resilience, durability, and/or recoverability under mechanical load. By way of non-limiting example, mechanical outcomes may include increases in tensile strength of about 1-100%, 2-80%, 5-50%, 5-28%, and/or any subrange and/or discrete value encompassed therein; increases in elasticity and/or elongation before break of about 5-150%, 10-120%, 20-100%, 40-70%, and/or any subrange and/or discrete value encompassed therein; reductions in breakage of about 5-90%, 10-70%, 20-50%, ≥30%, and/or any subrange and/or discrete value encompassed therein; improvements in hydration retention of about 1-100%, 5-60%, 10-40%, ≥15%, and/or any subrange and/or discrete value encompassed therein; and/or sustained curl pattern stability, shape retention, and/or resistance to humidity-induced deformation without chemical straightening, relaxing, and/or disulfide bond cleavage. These effects arise from internal, physical reinforcement and/or scaffold-mediated load distribution rather than chemical restructuring of keratin.

Upon formation within the cortex, the three-dimensional intrafiber scaffold occupies micro-scale interstitial spaces between keratin macrofibrils, providing internal reinforcement that stabilizes fiber geometry. In certain embodiments, such spatial occupancy reduces micro-buckling under tension, minimizes torsional collapse during styling, and maintains curl or wave formation by resisting uncontrolled shrinkage or over-expansion. Because the scaffold is hydration-responsive, it may expand and contract with changes in internal moisture content, preserving natural texture while reducing frizz and breakage.

In certain embodiments, the scaffold acts as a stress-distribution matrix in which polymerized lignin-rich domains provide semi-rigid reinforcement and starch-rich domains provide flexible damping zones. This hybrid internal network may absorb and redistribute mechanical forces along the fiber, reduce localized stress concentrations that trigger breakage, and stabilize keratin macrofibril bundles without restricting natural fiber motion.

In certain embodiments, curl modulation is achieved through physical structural reinforcement rather than chemical rearrangement of keratin bonds. Scaffold formation aligned with the natural curvature of the hair fiber may establish a stable internal geometry, while flexible domains within the scaffold permit elastic deformation and recovery during hydration-dehydration cycles. Such embodiments provide controlled modulation of curl behavior without disulfide bond cleavage, high-pH swelling, aldehydes, glyoxal, phenolic crosslinkers, or other relaxer chemistries.

In certain embodiments, the semi-flexible, hydration-responsive scaffold enables the hair fiber to resist excessive contraction during drying and to maintain curl pattern consistency after multiple wash cycles. Breakage during combing or detangling may be reduced, and elastic rebound may improve, resulting in more uniform and predictable curl behavior.

In certain embodiments, the structural reinforcement provided by the intrafiber scaffold is semi-permanent to long-lasting, depending on scaffold density, spatial distribution, penetration depth, degree of polymerization, frequency of application, hair porosity, and/or hair treatment history. In certain embodiments, the scaffold forms within the cortex at depths of about 0.1 μm to about 100 μm, 1 μm to about 50 μm, 2 μm to about 25 μm, 4 μm to about 18 μm, and/or any subrange and/or discrete value encompassed therein from the cuticle surface, and includes covalent and/or non-covalent linkages formed during lignin polymerization, association, and/or network formation, such that reinforcement may persist through multiple wash cycles and routine grooming. Such longevity is achieved without altering disulfide bonds, increasing pH to damaging levels, restructuring keratin chemistry, inducing oxidative bleaching, and/or causing cumulative chemical damage. In certain embodiments, the scaffold is biodegradable and may gradually remodel through normal oxidative or mechanical processes without irreversible chemical modification of keratin. Reapplication may strengthen results without inducing brittleness. In certain embodiments, controlled reversibility may be achieved by application of a terpenoid-containing solvent system (e.g., limonene in a cosmetic oil carrier) configured to disrupt non-covalent interactions within the polymerized lignin-starch network, permitting partial disassembly of the scaffold and restoration of natural curl behavior.

In some embodiments, the intrafiber scaffold may be configured as a responsive and/or “smart” network that changes mechanical properties in response to environmental stimuli. For example, scaffold density and/or flexibility may be tuned to respond to humidity, temperature, pH, and/or exposure to light, enabling dynamic control of curl pattern retention, frizz resistance, and/or volume in different environmental conditions. Such responsive scaffolds may incorporate moieties that reversibly expand and/or contract or alter intermolecular interactions under defined stimuli.

In certain embodiments, scaffold formation may be spatially controlled within the cortex to form gradients of scaffold density along the length and/or radius of a hair fiber. For example, a higher scaffold density may be formed near the cuticle to increase surface strength and abrasion resistance, while a lower density scaffold may be formed deeper within the cortex to preserve flexibility. Such programmable architectures may be achieved through controlled penetration depth, staged enzyme activation, or sequential application protocols.

In certain embodiments, the intrafiber scaffold forms a semi-flexible, dynamic nanolattice within the cortex that is capable of reversible mechanical deformation in response to bending, torsion, hydration, and drying cycles of the hair fiber. The scaffold network may elastically deform and recover in concert with native keratin macrofibrils, thereby permitting natural fiber motion while providing internal reinforcement. In some embodiments, the nanolattice may dynamically redistribute internal stress along curved regions of the hair fiber, including regions of elevated curvature, thereby reducing localized fracture initiation and promoting elastic recovery. Such dynamic behavior allows reinforcement without inducing stiffness, brittleness, or loss of natural movement.

In some embodiments, additional functional agents may be incorporated into and/or onto the composite nanoparticles prior to scaffold formation, enabling the intrafiber scaffold to serve as a reservoir or carrier for active compounds. For example, conditioning agents, antioxidants, UV-protective agents, fragrances, antimicrobial agents, and/or scalp-care actives may be co-localized within the scaffold to provide sustained or controlled release from within the hair fiber over time.

In some embodiments, the intrafiber scaffold may be engineered to be selectively reversible and/or degradable under predefined conditions, such as exposure to specific solvents, enzymes, pH shifts, and/or thermal treatments. This may enable controlled removal and/or remodeling of the scaffold to restore the hair fiber to a prior state, allowing reversible styling and/or periodic renewal of scaffold architecture without cumulative buildup.

In certain embodiments, the polymerized intrafiber scaffold forms a percolative nanoscale lattice or interconnected network within interstitial spaces of the cortex. The scaffold topology may include interconnected junctions and load-bearing pathways that distribute applied mechanical stress through the interior of the hair fiber. Such network architecture may provide dynamic mechanical reinforcement that accommodates hydration -dehydration cycles, flexural deformation, and tensile loading, thereby improving fiber resilience and curl stability without inducing rigidity or brittleness.

In certain embodiments, composite lignin-starch nanoparticles preferentially localize within regions of elevated curvature along the intrinsic curl path of a hair fiber, including localized curvature vertices corresponding to bends, kinks, or geometric maxima of the fiber. Such regions exhibit transient increases in cuticle interstice spacing during mechanical elongation, grooming, or mild thermal exposure, thereby facilitating preferential nanoparticle ingress and accumulation at mechanically stressed regions of the cortex.

In certain embodiments, penetration of composite lignin-starch nanoparticles preferentially occurs at localized coil-bend inflection regions along the intrinsic curl path of a hair fiber. Such regions, referred to herein as “Z-entry junctions,” correspond to localized angular transitions or inflection points in the fiber curvature that exhibit transient increases in cuticle interstice spacing during bending, hydration-dehydration cycles, grooming, or mild thermal exposure. These Z-entry junctions may function as preferential nanoparticle ingress sites that facilitate deeper cortical penetration relative to straighter fiber segments, thereby enhancing localized scaffold formation at mechanically stressed regions of the cortex.

In certain embodiments, ‘curvature vertices’refer to localized regions of increased geometric curvature along a hair fiber, including apex regions of curls, waves, bends, or torsional inflection points, which experience elevated mechanical stress during styling, hydration-dehydration cycles, and mechanical loading.

In certain embodiments, diagnostic and/or data-driven approaches may be incorporated to tailor scaffold formation parameters to individual hair characteristics. For example, treatment formulations and/or protocols may be customized based on hair diameter, porosity, curl pattern, damage level, and/or chemical treatment history. In certain embodiments, digital tools and/or sensor-based diagnostics may guide selection of nanoparticle concentration, enzyme activity, contact time, or treatment frequency to achieve personalized cosmetic outcomes.

In certain embodiments, enzyme-mediated polymerization/enzyme-activated polymerization of the lignin phase occurs preferentially at nanoparticle-rich curvature vertices within the cortex, resulting in the localized formation of a nanoscale reinforcing lattice configured to distribute tensile and bending stresses concentrated at such vertices. This vertex-selective reinforcement reduces initiation of microfractures and mitigates stress concentration effects associated with repeated mechanical deformation of curled or coiled hair fibers.

In certain embodiments, hair fibers exhibit localized regions of elevated curvature along their intrinsic curl path, including regions of maximum curvature, inflection points, and stress concentration zones that arise during hydration-dehydration cycles, mechanical manipulation, or styling. Such regions may be referred to herein as “curvature vertices” and are associated with localized mechanical strain, micro-buckling, torsional stress, and fracture initiation in untreated hair fibers. In certain embodiments, the intrafiber scaffold preferentially distributes mechanical load in and around such curvature vertices, reducing localized stress concentrations and stabilizing fiber geometry during bending, twisting, drying, and rehydration. Without being bound by theory, reinforcement of these regions contributes to improved curl cohesion, reduced frizz formation, reduced breakage, and improved resilience of highly curved or coiled hair textures.

In certain embodiments, the polymerized intrafiber scaffold formed at curvature vertices comprises a dynamic nanolattice that exhibits semi-flexible deformation under applied strain. The nanolattice accommodates reversible elongation and recovery of the hair fiber without inducing brittleness, enabling enhanced elasticity while preserving natural movement of the fiber.

In certain embodiments, preferential scaffold formation at Z-entry junctions reduces localized tensile and torsional stress concentrations at condensed coil or bend regions during elongation and styling. Reinforcement of such regions redistributes mechanical load away from high-curvature zones, thereby reducing fracture initiation, micro-buckling, and fatigue-related damage during repeated mechanical deformation of coiled and highly curved hair fibers.

In certain embodiments, scaffold-forming compositions may be delivered using specialized application devices designed to enhance penetration, distribution, and/or activation. Such devices may include applicators with controlled heating, micro-misting systems, vibration and/or ultrasonic assistance, and/or controlled oxygen delivery to modulate enzyme activity. Device-assisted delivery may be particularly beneficial for achieving uniform scaffold formation along long hair fibers and/or in high-density hair regions.

In certain embodiments, prior to polymerization, spherical lignin-starch nanostructures exhibit rotational mobility within an aqueous carrier, enabling ball-bearing-like gliding along curved cuticle interfaces. This rotational mobility facilitates nanoparticle transport through cuticle interstices at curvature vertices with reduced friction and without cuticle lifting or delamination.

In some embodiments, the intrafiber scaffold platform may be extended to additional keratinous and/or fibrous substrates beyond human scalp hair, including eyelashes, eyebrows, animal fur, wool, and/or other natural fibers. Further extensions may apply the scaffold concept to non-keratin fibrous substrates, such as cellulose-based textiles and/or biopolymer fibers, for applications in material reinforcement, protective coatings, and/or durability enhancement.

In certain embodiments, prior to polymerization, spherical lignin-starch nanostructures exhibit rotational mobility within an aqueous carrier, enabling ball-bearing-like gliding along curved cuticle interfaces. This rotational mobility facilitates nanoparticle transport through cuticle interstices at curvature vertices with reduced friction and without cuticle lifting or delamination.

In some embodiments, the composite nanoparticles and scaffold materials may be sourced from circular and/or upcycled biomass streams, such as agricultural byproducts and/or forestry waste, to enhance sustainability. Scaffold systems may be engineered for improved biodegradability and/or environmental compatibility following washing and/or disposal, enabling eco-conscious cosmetic and material treatment products.

In certain embodiments, the enzyme system comprises a phenol oxidase enzyme, such as laccase present at an activity of about 0.001-1,000 U/mL, 0.01-500 U/mL, 0.1-200 U/mL, 0.5-100 U/mL, 1-50 U/mL, 2-40 U/mL, 5-30 U/mL, 10-25 U/mL, and/or any subrange and/or discrete value encompassed therein, in the formulation at a loading of about 0.0001-20 wt %, 0.001-10 wt %, 0.005-5 wt %, 0.01-2.0 wt %, 0.02-1.0 wt %, 0.05-0.8 wt %, 0.1-0.5 wt %, and/or any subrange and/or discrete value encompassed therein based on total formulation weight. In further embodiments, other phenol-oxidizing enzymes, including, but not limited to, peroxidases, tyrosinases, catechol oxidases, and/or enzyme-mediator systems, may be used alone and/or in combination with a phenol oxidase enzyme, such as laccase at comparable activity ranges, and/or any subrange and/or discrete value encompassed therein.

In certain embodiments, redox mediators, electron shuttles, and/or radical transfer agents may be included to facilitate oxidative coupling of lignin phenolic moieties, including, but not limited to, natural and/or synthetic mediator compounds compatible with cosmetic use and enzyme stability. By way of non-limiting example, mediator compounds may be present at concentrations in a range of about 0.001 μM to about 500 mM, 0.01 μM to about 200 mM, 0.1 μM to about 50 mM, 1 μM to about 10 mM, 5 μM to about 5 mM, 10 μM to about 1 mM, and/or any subrange and/or discrete value encompassed therein.

In certain embodiments, oxygen availability, redox environment, and/or dissolved gas content is controlled and/or modulated to tune polymerization kinetics, penetration timing, scaffold density, and/or network architecture. By way of non-limiting example, formulations may be prepared, stored, and/or packaged under reduced-oxygen, inert gas, vacuum, and/or modified-atmosphere conditions and activated upon exposure to ambient air during application, handling, mixing, and/or spreading, and/or oxygen scavengers, antioxidants, and/or redox buffers may be included at concentrations of about 0.0001-10 wt %, 0.001-5 wt %, 0.01-2 wt %, 0.01-1.0 wt %, 0.05-0.8 wt %, and/or any subrange and/or discrete value encompassed therein to delay polymerization until after intrafiber localization. In further embodiments, oxygen-permeable applicators, packaging, films, membranes, and/or delivery devices may be used to regulate oxygen exposure, diffusion, and/or activation during use.

In certain embodiments, polymerization onset following intrafiber localization occurs within about 1 second to about 60 minutes, 5 seconds to about 30 minutes, 30 seconds to about 10 minutes, 1 minute to about 15 minutes, 2 minutes to about 12 minutes, and/or any subrange and/or discrete value encompassed therein after activation, and polymerization may proceed over a duration of about 10 seconds to about 24 hours, 1 minute to about 4 hours, 2 minutes to about 120 minutes, 2 minutes to about 30 minutes, 5 minutes to about 20 minutes, and/or any subrange and/or discrete value encompassed therein depending on enzyme activity, mediator concentration, oxygen availability, temperature, nanoparticle loading, formulation rheology, and/or application conditions. In certain embodiments, reaction kinetics may be modulated, quenched, slowed, accelerated, and/or staged to avoid over-polymerization and to tune scaffold density, spatial distribution, connectivity, and/or mechanical response within the cortex.

In certain embodiments, enzyme-mediated polymerization/enzyme-activated polymerization of lignin phenolic moieties is modulated by controlling oxygen availability, redox environment, and formulation buffering conditions. For example, oxygen scavengers, buffering agents, or controlled-permeability packaging may be employed to delay oxidative polymerization during storage and transport, with polymerization activated upon exposure to ambient oxygen during application. In further embodiments, oxygen-permeable applicators, headspace control, or staged activation protocols may be used to regulate the rate and extent of polymer cross-linking within the cortex. Such modulation permits controlled scaffold density, avoids over-polymerization, and enables tuning of mechanical reinforcement outcomes.

### Detailed Method of Manufacture

In certain embodiments, the methods of making and/or manufacturing described herein may be performed in any order, sequence, and/or combination, and one or more steps may be added, omitted, repeated, subdivided, and/or combined without departing from the scope of the invention. The following description is illustrative and non-limiting.

### A. Preparation of Composite Lignin-Starch Nanoparticles.

In certain embodiments, composite lignin-starch nanoparticles are prepared by dispersing a plant-derived lignin phase and/or a starch phase in an aqueous, hydroalcoholic, and/or mixed solvent medium under controlled temperature, shear, and/or mixing conditions. The lignin phase may be provided as organosolv lignin, kraft lignin, soda lignin, lignosulfonates, fractionated lignin, modified lignin, and/or combinations thereof, and the starch phase may be provided as corn starch, potato starch, tapioca starch, rice starch, modified starches, dextrins, amylose-rich fractions, amylopectin-rich fractions, and/or combinations thereof.

In certain embodiments, scaffold density may be selectively reduced, modified, remodeled, and/or partially reversed using solvent systems, including, but not limited to, terpenoid-containing compositions, essential oil fractions, and/or hydrophobic carrier systems, configured to disrupt non-covalent interactions within the polymerized lignin-starch scaffold without cleaving keratin disulfide bonds.

The lignin and starch components are combined at predetermined and/or adjustable weight ratios selected to achieve desired nanoparticle rigidity, dispersibility, surface charge, penetration behavior, and/or enzyme accessibility. The combined dispersion is subjected to controlled agitation, sonication, homogenization, microfluidization, high-pressure emulsification, rotor-stator mixing, and/or other high-shear processing techniques to form composite nanoparticles having an average diameter in a range of about 1 nm to about 5,000 nm, 10 nm to about 2,000 nm, 20 nm to about 1,000 nm, 50 nm to about 500 nm, 60 nm to about 400 nm, 80 nm to about 250 nm, 100 nm to about 200 nm, and/or any subrange and/or discrete value encompassed therein. By way of non-limiting example, in certain embodiments, average nanoparticle diameters may be about 50 nm, 75 nm, 100 nm, 125 nm, 150 nm, 175 nm, 200 nm, 250 nm, 300 nm, 350 nm, 400 nm, 450 nm, and/or any combination thereof. In certain embodiments, narrower subranges may be selected based on penetration depth, scaffold formation kinetics, and/or target hair type.

In certain embodiments, surfactant-free, low-surfactant, and/or polymer-stabilized conditions are employed to maintain cosmetic compatibility, reduce residue on hair fibers, and/or improve intrafiber delivery efficiency.

The resulting nanoparticle dispersion may be subjected to size classification, fractionation, filtration, centrifugation, membrane separation, and/or other post-processing techniques to narrow, tailor, and/or control particle size distribution. The nanoparticles may be freeze-dried, spray-dried, vacuum-dried, and/or otherwise dehydrated to form a dry or semi-dry powder for improved shelf stability. Where freeze-drying is employed, cryoprotectants, lyoprotectants, sugars, polyols, and/or stabilizers may be added to preserve nanoparticle morphology and redispersibility upon rehydration.

### B. Incorporation of Enzyme System

In certain embodiments, a phenol oxidase enzyme, such as laccase enzyme and/or other phenol-oxidizing enzyme, is prepared in an aqueous buffer and/or cosmetically acceptable medium compatible with enzyme stability and activity. The enzyme solution may be formulated with stabilizers, antioxidants, chelators, preservatives, and/or excipients selected to maintain activity during storage, transport, and/or handling. In certain embodiments, the enzyme is provided separately from the nanoparticle composition to delay polymerization until after intrafiber localization.

In alternative embodiments, the enzyme system may be co-formulated with the composite nanoparticles in a single composition, provided that formulation conditions are selected and/or adjusted to suppress premature polymerization during storage, including, but not limited to, controlling oxygen availability, temperature, moisture content, redox environment, and/or inclusion of reversible enzyme inhibitors and/or oxygen scavengers.

### C. Formulation of Cosmetic Treatment Composition

In certain embodiments, the composite nanoparticles and enzyme system are combined with a cosmetically acceptable carrier to form a treatment composition having a pH in a range of about 4.0 to about 8.5, 4.5 to about 7.5, 5.0 to about 6.8, 5.2 to about 6.5, 5.5 to about 6.2, 5.5 to about 6.0, and/or any subrange and/or discrete value encompassed therein. By way of non-limiting example, pH values may include about 5.0, 5.1, 5.2, 5.3, 5.4, 5.5, 5.6, 5.7, 5.8, 5.9, 6.0, 6.1, 6.2, 6.3, 6.4, and/or 6.5. Additional cosmetic excipients, including, but not limited to, humectants, thickeners, rheology modifiers, conditioners, fragrances, preservatives, chelators, UV stabilizers, and/or film-formers, may be added provided that such excipients do not substantially inhibit enzyme activity and/or nanoparticle penetration.

The formulation may be prepared in various cosmetic formats, including, but not limited to, lotions, creams, gels, foams, sprays, serums, emulsions, microemulsions, and/or encapsulated delivery systems. The viscosity and rheology of the formulation may be adjusted and/or tuned to promote uniform coating of hair fibers and adequate residence time to allow nanoparticle penetration into the cortex prior to scaffold formation and/or polymerization.

### D. Kit Assembly and Packaging

In kit embodiments, the composite nanoparticles are packaged in a first container, optionally in dry, freeze-dried, or rehydratable form, and the enzyme system is packaged in a second container. Instructions for reconstitution, mixing, and/or application may be provided. Optional pretreatment compositions, post-treatment sealants, shampoos, conditioners, applicators, mixing vessels, oxygen-permeable applicators, and/or packaging components may be included. Packaging configurations may be selected for professional salon use, consumer home use, single-use formats, multi-use formats, and/or any combination thereof.

In certain embodiments, processing parameters including shear rate, homogenization energy, sonication intensity, polymer phase ratio, and solvent conditions may be selected to promote formation of generally spherical or quasi-spherical composite nanoparticles. Control of such parameters may influence domain organization, surface topology, and internal morphology of the nanoparticles, thereby affecting cortical penetration behavior and intrafiber scaffold architecture.

### Detailed Method of Use

In certain embodiments, the methods of use described herein may be performed in any order, sequence, and/or combination, and one or more steps may be added, omitted, repeated, subdivided, and/or combined without departing from the scope of the invention. The following description is illustrative and non-limiting.

### A. Hair Preparation

Prior to treatment, the hair may be cleansed, clarified, preconditioned, and/or otherwise prepared to remove oils, residues, styling products, environmental contaminants, and/or sebum that could impede nanoparticle wetting, deposition, and/or penetration. In certain embodiments, the hair is washed with a mild shampoo, clarifying shampoo, and/or surfactant-containing cleanser and towel-dried, air-dried, or partially dried to a damp, moist, or semi-dry state. In other embodiments, the scaffold-forming composition may be applied to dry, partially dry, and/or pre-hydrated hair to leverage hygroscopic uptake of the formulation into the fiber, thereby enhancing nanoparticle transport through cuticle interstices into the cortex. Pretreatment compositions, primers, wetting agents, chelators, pH adjusters, and/or conditioning compositions may be applied to adjust surface properties of the cuticle, modulate ionic strength, and/or promote uniform wetting and deposition of the formulation on the hair fibers.

### B. Application of Nanoparticle Composition

The composite lignin-starch nanoparticle composition is applied to the hair by any suitable delivery technique, including, but not limited to, brushing, spraying, misting, foaming, combing, massaging, saturating, sectioning, and/or coating the formulation onto the hair fibers. The formulation may be distributed along the length of the hair fibers, at selected regions, and/or in patterned application to promote uniform and/or targeted coverage. The nanoparticles are allowed to migrate, diffuse, and/or be transported through cuticle interstices into the cortex region of the hair fibers during a penetration phase. In certain embodiments, penetration is facilitated by maintaining the hair in a moist, hydrated, or semi-hydrated state; gently warming the hair to cosmetically acceptable temperatures; and/or applying mild mechanical action including, but not limited to, combing, brushing, finger manipulation, vibration, and/or low-tension stretching. In certain embodiments, mild mechanical tension (including, but not limited to, combing, brushing, or finger-tension) may be applied during distribution to transiently increase cuticle flare and/or lamellar spacing in regions of curvature, thereby facilitating nanoparticle entry into the cortex. Penetration times may be selected and/or adjusted based on hair thickness, diameter, porosity, curl pattern, treatment history, formulation parameters, and/or desired scaffold density.

### C. Activation of Enzyme and Scaffold Formation

Following the penetration phase, the enzyme system, including a phenol oxidase enzyme, such as laccase and/or other phenol-oxidizing enzymes, is applied, activated, and/or allowed to initiate oxidative polymerization of phenolic moieties of the lignin phase within the cortex. Polymerization may occur under ambient oxygen, controlled oxygen, and/or modified atmospheric conditions and at temperatures compatible with cosmetic use, including temperatures of about 5° C. to about 80° C., 10° C. to about 60° C., 20° C. to about 55° C., 25° C. to about 50° C., 29° C. to about 45° C., 30° C. to about 40° C., and/or any subrange and/or discrete value encompassed therein. In certain embodiments, enzyme activation and scaffold formation are promoted by mild heat using cosmetically compatible heat sources including, but not limited to, hooded dryers, handheld dryers, infrared emitters, steam tools, heated caps, thermal wraps, and/or warming devices. In embodiments in which the enzyme is co-formulated with the nanoparticles, polymerization may be initiated and/or accelerated by exposure to air, moisture, oxygen, and/or mild warming. As polymerization proceeds, the composite nanoparticles interconnect to form a polymerized intrafiber scaffold within the cortex that is physically distinct from the native keratin protein network and reinforces the hair fiber without cleaving disulfide bonds. In certain embodiments, the composition is maintained on the hair for a duration of about 30 seconds to about 24 hours, 2 minutes to about 120 minutes, 5 minutes to about 60 minutes, 10 minutes to about 45 minutes, 15 minutes to about 40 minutes, and/or any subrange and/or discrete value encompassed therein to allow penetration, polymerization, and/or scaffold formation, with duration optionally adjusted based on hair type, porosity, and/or desired scaffold density.

### D. Post-Treatment Conditioning and Finishing

After scaffold formation, the hair may be rinsed, blotted, wiped, and/or left unrinsed to remove residual formulation from the cuticle surface, depending on formulation design and desired cosmetic outcome. A post-treatment conditioner, sealant, coating composition, and/or styling formulation may be applied to condition the hair surface, enhance tactile feel, manageability, gloss, and/or provide additional cosmetic benefits. The system may be configured as a rinse-off treatment, a leave-in reinforcement, and/or a hybrid protocol comprising a partial rinse followed by a secondary leave-in booster formulation. In certain embodiments, leave-in products may be used to maintain moisture, reduce friction, and/or protect the treated hair fibers from environmental exposure, UV radiation, humidity, and/or mechanical wear.

### E. Reapplication and Maintenance

In certain embodiments, the treatment may be reapplied periodically, intermittently, and/or as part of a maintenance regimen to maintain, rebuild, and/or progressively increase intrafiber scaffold density over time. Treatment frequency may be selected based on hair condition, porosity, prior chemical or thermal treatment history, desired level of reinforcement, and/or cosmetic outcomes. In certain embodiments, partial degradation, remodeling, and/or redistribution of the scaffold may occur naturally over time through washing, grooming, and environmental exposure, allowing subsequent treatments to renew, reinforce, and/or adjust scaffold formation without inducing cumulative damage, brittleness, and/or loss of flexibility.

In certain embodiments, the methods of use described herein may be performed in any order, sequence, and/or combination, and one or more steps may be added, omitted, repeated, subdivided, overlapped, and/or combined without departing from the scope of the invention. By way of non-limiting example, pretreatment, wetting, penetration, enzyme activation, polymerization, rinsing, conditioning, sealing, and/or finishing steps may be performed in alternative sequences, in parallel, iteratively, and/or in staged cycles depending on formulation format, hair type, curl pattern, porosity, treatment objectives, and/or application context. In certain embodiments, one or more optional steps may be omitted, and in other embodiments, additional steps may be included, provided that formation of an intrafiber scaffold within the cortex is achieved.

### A. Hair Preparation

Prior to treatment, the hair may be cleansed, clarified, preconditioned, and/or otherwise prepared to remove oils, residues, styling products, environmental contaminants, and/or sebum that could impede nanoparticle wetting, deposition, and/or penetration. In certain embodiments, the hair is washed with a mild shampoo, clarifying shampoo, and/or surfactant-containing cleanser and towel-dried, air-dried, or partially dried to a damp, moist, semi-dry, and/or dry state. Pretreatment compositions, primers, wetting agents, chelators, pH adjusters, and/or conditioning compositions may be applied to adjust surface properties of the cuticle, modulate ionic strength, and/or promote uniform wetting and deposition of the formulation on the hair fibers.

### B. Application of Nanoparticle Composition

The composite lignin-starch nanoparticle composition is applied to the hair by any suitable technique, including, but not limited to, brushing, spraying, misting, foaming, combing, massaging, saturating, sectioning, coating, and/or targeted placement of the formulation onto the hair fibers. The formulation may be distributed along the length of the hair fibers, at selected regions, and/or in patterned application to promote uniform and/or localized coverage. The nanoparticles are allowed to migrate, diffuse, and/or be transported through cuticle interstices into the cortex region of the hair fibers during a penetration phase.

In certain embodiments, penetration is facilitated by maintaining the hair in a moist, hydrated, and/or semi-hydrated state; gently warming the hair to cosmetically acceptable temperatures; and/or applying mild mechanical action including, but not limited to, combing, brushing, finger manipulation, vibration, and/or low-tension stretching. Penetration times and/or conditions may be selected and/or adjusted based on hair thickness, diameter, porosity, curl pattern, prior chemical and/or thermal treatment history, formulation parameters, and/or desired scaffold density and spatial distribution.

### C. Activation of Enzyme and Scaffold Formation

Following the penetration phase, the enzyme system, including a phenol oxidase enzyme, such as laccase and/or other phenol-oxidizing enzymes, is applied, activated, and/or allowed to initiate oxidative polymerization of phenolic moieties of the lignin phase within the cortex. Polymerization may occur under ambient oxygen, controlled oxygen, and/or modified atmospheric conditions and at temperatures compatible with cosmetic use. In embodiments in which the enzyme is co-formulated with the nanoparticles, polymerization may be initiated and/or accelerated by exposure to air, moisture, oxygen, and/or mild warming.

As polymerization proceeds, the composite nanoparticles interconnect, associate, aggregate, and/or polymerize to form a polymerized intrafiber scaffold within the cortex. The scaffold is physically distinct from the native keratin protein network and reinforces the hair fiber without cleaving disulfide bonds. The duration and/or extent of the polymerization phase may be selected and/or adjusted to tune scaffold density, spatial distribution, and/or resulting mechanical properties of the treated hair fibers.

In certain embodiments, organization of nanoparticles into intrafiber networks may occur through a combination of enzyme-mediated covalent coupling and non-covalent association, including hydrogen bonding, π-π interactions of lignin domains, hydrophobic association, and/or physical confinement within cortical microenvironments.

In certain embodiments, polymerization of the lignin phase and/or formation of the intrafiber scaffold occur predominantly within the cortex at depths of about 0.1 μm to about 100 μm, 1 μm to about 50 μm, 2 μm to about 25 μm, 4 μm to about 18 μm, and/or any subrange and/or discrete value encompassed therein from the outer cuticle surface, while remaining substantially excluded from the medulla.

### D. Post-Treatment Conditioning and Finishing.

After scaffold formation, the hair may be rinsed, blotted, wiped, and/or left unrinsed to remove residual formulation from the cuticle surface. A post-treatment conditioner, sealant, coating composition, and/or styling formulation may be applied to condition the hair surface, enhance tactile feel, manageability, gloss, and/or provide additional cosmetic benefits. In certain embodiments, leave-in products may be used to maintain moisture, reduce friction, and/or protect the treated hair fibers from environmental exposure, UV radiation, humidity, and/or mechanical wear.

### E. Reapplication and Maintenance.

In certain embodiments, the treatment may be reapplied periodically, intermittently, and/or as part of a maintenance regimen to maintain, rebuild, and/or progressively increase intrafiber scaffold density over time. Treatment frequency may be selected and/or adjusted based on hair condition, porosity, prior chemical and/or thermal treatment history, desired level of reinforcement, and/or cosmetic outcomes. In certain embodiments, partial degradation, remodeling, and/or redistribution of the scaffold may occur naturally over time through washing, grooming, and environmental exposure, allowing subsequent treatments to renew, reinforce, and/or adjust scaffold formation.

### Scaffold Formation and Structural Architecture (Additional Patent-Safe Embodiments)

Upon reaching the cortex, the composite lignin-starch nanoparticles may undergo self-organization, association, aggregation, and/or enzyme-mediated polymerization/ enzyme-activated polymerization to form a biodegradable intrafiber scaffold that reinforces the native keratin matrix without chemically modifying keratin disulfide bonds. In certain embodiments, the scaffold is non-crystalline, semi-flexible, hydration-responsive, and compatible with the hygroscopic environment of the hair fiber, enabling mechanical reinforcement while preserving natural fiber motion, elasticity, and texture.

In certain embodiments, differences in hydrophilicity between lignin-rich domains and starch-rich domains promote spatial organization within the cortex, with lignin-enriched regions preferentially associating with relatively lower-water microenvironments and starch-enriched regions orienting toward more hydrated regions. Such domain organization may promote cohesive aggregation and formation of interconnected scaffold domains without forcing displacement of keratin microfibrils.

In certain embodiments, enzyme-triggered oxidative polymerization of lignin phenolic moieties results in formation of new covalent and/or non-covalent linkages, including, but not limited to, C—C, C—O—C, ether, aryl-aryl, and/or aryl-alkyl linkages, within the cortex, causing localized fusion, association, and/or interconnection of composite nanoparticles into a continuous, semi-continuous, and/or percolating three-dimensional intrafiber network. Polymerization and/or network formation may be activated, initiated, and/or modulated by ambient oxygen, controlled oxygen exposure, endogenous moisture within the cortex, hydration state of the hair fiber, formulation water content, and/or external humidity, under cosmetically acceptable temperatures and pH conditions, and/or any combination thereof.

In certain embodiments, the resulting intrafiber scaffold comprises an interconnected three-dimensional mesh or network that is semi-porous and mechanically compliant, allowing continued moisture exchange while providing internal mechanical reinforcement. The scaffold may be oriented, in part, along the natural macrofibrillar organization of keratin within the cortex, thereby distributing mechanical stress and reducing localized strain during bending, stretching, or styling.

In certain embodiments, scaffold maturation occurs through a combination of moisture-assisted expansion of starch-rich domains, mild thermally enhanced molecular mobility, and time-dependent gelation during drying, resulting in a transition from dispersed nanoparticles to a mechanically supportive composite network. The scaffold remains adaptable and responsive to hydration cycles, expanding and contracting with changes in internal moisture content without inducing brittleness.

In certain embodiments, treated hair fibers exhibit measurable improvements in one or more mechanical and/or functional properties relative to untreated controls under comparable test conditions, including, but not limited to, increases in tensile strength of about 1-100%, 2-80%, 5-50%, 5-28%, and/or any subrange and/or discrete value encompassed therein; increases in elongation before break of about 5-150%, 10-120%, 20-100%, 40-70%, and/or any subrange and/or discrete value encompassed therein; improvements in hydration buffering and/or moisture retention of about 1-100%, 5-60%, 10-40%, ≥15%, and/or any subrange and/or discrete value encompassed therein; and reductions in breakage, fracture, and/or fiber failure of about 5-90%, 10-70%, 20-50%, ≥30%, and/or any subrange and/or discrete value encompassed therein, as measured by tensile testing, cyclic fatigue testing, humidity exposure protocols, and/or other suitable mechanical and/or environmental test methods.

In certain embodiments, polymerization kinetics are influenced by local oxygen availability, moisture content, and temperature within the cortex, such that enzyme-mediated inter-nanoparticle crosslink formation preferentially occurs after cortical penetration rather than at the cuticle surface.

The disclosed compositions, methods, and kits are distinguished from known cosmetic, consumer, and professional hair treatment systems by the formation of a biodegradable intrafiber scaffold within the cortex via delivery of composite lignin-starch nanoparticles and enzyme-triggered polymerization. In contrast to surface-coating approaches and/or keratin-reactive chemistries, the systems described herein strengthen and/or modify hair primarily through physical internal reinforcement rather than chemical modification of keratin, manipulation of disulfide bonds, and/or formation of external films.

Bond-building systems, including, but not limited to, bis-amines, thiol-based products, peptide-based systems, carbamate chemistries, and/or related keratin-reactive formulations, operate by forming, repairing, rearranging, supplementing, and/or interacting with chemical bonds within keratin and/or by engaging directly with reactive keratin functional groups. In contrast, the present invention does not rely on repair, rearrangement, supplementation, and/or direct chemical modification of disulfide, hydrogen, ionic, and/or peptide bonds of keratin. Instead, the systems described herein introduce a biodegradable three-dimensional intrafiber scaffold within the cortex that distributes mechanical stress, stabilizes internal architecture, and/or reinforces the keratin matrix without altering keratin chemistry.

Non-caustic straightening, smoothing, and/or reshaping systems, including, but not limited to, glyoxylic acid systems, carbocysteine systems, amino acid complexes, aldehyde-based treatments, and/or heat-activated crosslinking formulations, rely on heat-induced crosslinking, protein denaturation, aldehyde-like reactions, and/or chemical realignment of keratin. The present invention does not engage in peptide bond cleavage, glyoxylation, aldehyde chemistry, formaldehyde-releasing reactions, and/or chemical realignment of keratin. Curl modulation, shape retention, and/or smoothing effects, when present, arise from internal physical reinforcement and/or scaffold-mediated stress distribution rather than chemical smoothing, protein restructuring, and/or keratin denaturation. Enzyme activation and scaffold formation occur under cosmetically acceptable temperature and pH conditions that preserve keratin integrity.

Conventional relaxers and high-pH treatments operate by swelling hair fibers, opening the cuticle, and cleaving disulfide bonds under strongly alkaline conditions. In contrast, the disclosed compositions operate under mildly acidic to near-neutral pH conditions and do not induce chemical swelling, aggressive cuticle lifting, and/or disulfide bond cleavage. Structural reinforcement is achieved through intrafiber scaffold formation rather than irreversible chemical restructuring of keratin.

Surface conditioners, film-formers, and coating-based systems, including, but not limited to, silicones, quaternary ammonium compounds, polyquaterniums, proteins, waxes, oils, and/or polymer films, primarily deposit materials on the cuticle and provide temporary smoothing, lubrication, and/or gloss. In contrast, the present invention enables penetration into the cortex and in situ polymerization to form a durable internal scaffold that provides mechanical reinforcement, durability, and/or shape stabilization beyond surface-level effects.

Nanotechnology-based cosmetic serums, liposomal conditioners, microemulsion delivery systems, and/or carrier-based formulations primarily enhance delivery of active agents without forming durable internal structures. The present invention uniquely combines nanoparticle delivery with enzyme-triggered polymerization within the cortex to create a persistent, semi-permanent, and/or remodelable intrafiber scaffold that reinforces the hair fiber from within.

In certain embodiments, the disclosed technology is characterized by one or more of the following features, alone and/or in combination: (i) enzyme-activated intrafiber polymerization occurring after cortical penetration of composite nanoparticles; (ii) formation of a biodegradable internal scaffold that distributes mechanical stress without chemical modification of keratin; (iii) operation under non-caustic pH and cosmetically acceptable temperature conditions; and/or (iv) semi-permanent strengthening, curl modulation, and/or shape retention achieved through internal architectural reinforcement rather than chemical straightening, keratin-reactive chemistries, and/or surface coating. These features, alone and in combination, distinguish the disclosed systems and methods from known hair repair, conditioning, strengthening, smoothing, and straightening technologies.

### EXAMPLES

The following examples are provided to illustrate representative and non-limiting embodiments of the compositions, methods, and kits described herein. The examples are not intended to limit the scope of the invention, and variations in formulation, processing conditions, application protocols, hair type, and environmental conditions may be employed without departing from the scope of the invention.

### Example 1—Strengthening of Chemically Treated Hair Fibers

Human scalp hair fibers previously subjected to chemical coloring, bleaching, perming, relaxing, and/or thermal styling were washed with a mild shampoo, clarifying shampoo, and/or surfactant-containing cleanser and towel-dried, air-dried, and/or partially dried to a damp or semi-dry state. A composition comprising composite lignin-starch nanoparticles and a phenol oxidase enzyme, such as laccase enzyme in an aqueous carrier at a pH in a range of about 5.2 to about 6.5 was applied uniformly along the length of the hair fibers. The treated hair was maintained at ambient and/or mildly elevated temperature (e.g., about 20° C. to about 45° C.) for a duration of about 2 minutes to about 60 minutes to allow nanoparticle penetration into the cortex, followed by continued exposure to air, oxygen, and/or controlled humidity to permit enzyme-activated polymerization/enzyme-mediated polymerization of the lignin phase within the cortex.

After treatment, the hair fibers were rinsed, blotted, and/or left unrinsed and conditioned with a post-treatment conditioner, sealant, and/or styling formulation. Mechanical testing of treated hair fibers demonstrated increased tensile strength, improved elongation before break, and reduced breakage relative to untreated control fibers. The treated hair exhibited improved flexibility, reduced surface roughness, and maintained cuticle integrity.

### Example 2—Curl Pattern Retention Under High Humidity

Curly, coily, and/or wavy human hair fibers were treated with a composite lignin-starch nanoparticle composition and enzyme system as described in Example 1. Following scaffold formation within the cortex, the hair fibers were styled into defined curls, coils, waves, and/or shapes and allowed to dry under ambient conditions. The treated hair and untreated control hair were then exposed to a high-humidity environment (e.g., relative humidity of about 60% to about 95%) for several hours to several days.

The treated hair fibers exhibited improved curl pattern retention, reduced frizz formation, and reduced humidity-induced deformation relative to untreated control hair. The intrafiber scaffold provided internal mechanical reinforcement that reduced fiber collapse, shape relaxation, and/or moisture-induced swelling without stiffening the hair or causing brittleness.

### Example 3—Simultaneous Structural Reinforcement and Color Deposition

A chromogenic embodiment of the composition was prepared in which one or more cosmetically acceptable pigments, dyes, and/or colorants were associated with composite lignin-starch nanoparticles. The pigmented nanoparticle composition and enzyme system were applied to virgin and/or previously colored human hair fibers as described above. After penetration and enzyme-activated or enzyme-mediated polymerization/enzyme-activated polymerization, the hair was rinsed, conditioned, and/or styled.

The treated hair fibers exhibited uniform intrafiber color deposition along the cortex in addition to improved mechanical properties. The color remained stable after multiple wash cycles relative to conventional surface-deposited dyes, indicating intrafiber retention of pigment within the polymerized scaffold.

### Example 4—Repair of Heat-Damaged Hair with Improved Elasticity

Human hair fibers exhibiting signs of thermal damage from repeated blow-drying, flat-ironing, curling iron use, and/or other heat styling were treated using a multi-step kit comprising a pretreatment cleanser, a composite lignin-starch nanoparticle treatment composition, and a post-treatment sealant. The nanoparticle composition was allowed to penetrate into the cortex prior to activation of the enzyme system to form the intrafiber scaffold.

Post-treatment analysis demonstrated improved elasticity, reduced brittleness, and improved resistance to mechanical fatigue of the treated hair fibers relative to untreated damaged hair. The treated hair exhibited improved combability, reduced split-end formation, and improved tactile feel over repeated grooming cycles.

### Example 5—Professional Salon Application Protocol

A professional salon protocol was implemented in which a stylist applied the composite nanoparticle composition to freshly washed, towel-dried, and/or preconditioned hair of a client seeking reduced breakage, improved manageability, curl stabilization, and/or strength enhancement. After a penetration period of about 2 minutes to about 20 minutes, an enzyme activator composition was applied and allowed to react for about 1 minute to about 15 minutes. The hair was then rinsed, conditioned, and styled as desired.

The client reported improved softness, reduced tangling, enhanced durability of the hairstyle, and improved resistance to humidity over subsequent days and wash cycles. The hair maintained flexibility and natural movement while exhibiting reduced mechanical damage during brushing and styling.

### Example 6—Repeated Maintenance Treatments

Human hair fibers were subjected to an initial scaffold-forming treatment as described in Example 1. After a period of routine washing, environmental exposure, and styling (e.g., about 1 to about 8 weeks), a maintenance treatment was applied using a reduced concentration of composite nanoparticles and enzyme system. The maintenance treatment restored and/or augmented mechanical reinforcement of the hair fibers and maintained improved tensile strength, elasticity, and breakage resistance over multiple wash cycles.

### Example 7—Fine, Low-Porosity Hair Treatment

Fine, low-porosity human hair fibers were treated using a lower-viscosity formulation and reduced nanoparticle concentration to minimize surface residue while enabling cortical penetration. The treated hair exhibited improved strength and manageability without heaviness, stiffness, or buildup.

### Example 8—Coarse, High-Porosity Hair Treatment

Coarse, high-porosity human hair fibers were treated using an increased nanoparticle concentration and extended penetration time. The treated hair exhibited improved moisture retention, reduced breakage, and improved curl uniformity under humid conditions.

### Experimental Support and Prototype Embodiments

The following prototype embodiments, experimental observations, and representative evaluations are provided to further illustrate enablement and practical implementation of the compositions, methods, and kits described herein. These examples are illustrative and non-limiting, and variations in formulation, processing conditions, hair type, and evaluation methods may be employed without departing from the scope of the invention.

In certain embodiments, proof-of-concept prototype formulations of composite lignin-starch nanoparticles were prepared and evaluated to assess nanoparticle stability, penetration behavior, and enzyme-activated scaffold formation under cosmetically acceptable conditions. In representative prototype formulations, the composite nanoparticles exhibited average diameters in a range of about 60 nm to about 200 nm, optionally about 80 nm to about 160 nm, and in some embodiments about 98 nm to about 140 nm, with reduced agglomeration and improved suspension stability in aqueous and/or emulsion-based cosmetic carriers relative to larger particle populations. In certain embodiments, formulations were configured to maintain colloidal stability during storage and handling, with formulation viscosity adjusted to a range of about 200 cP to about 5,000 cP, optionally about 500 cP to about 1,500 cP, prior to enzyme activation to facilitate uniform distribution on hair fibers and controlled penetration.

In certain embodiments, enzyme-mediated polymerization/enzyme-activated polymerization of the lignin phase within the cortex was observed to occur under mild thermal activation conditions in a range of about 20° C. to about 45° C., optionally about 25° C. to about 40° C., and in some embodiments about 29° C. to about 35° C., consistent with cosmetically acceptable processing temperatures. Such activation conditions are compatible with typical salon and/or consumer heat sources and enable scaffold formation without inducing thermal damage to the cuticle or cortex. In some embodiments, lignin fractions having relatively lower phenolic content were employed to moderate polymerization kinetics and cosmetic transparency, while in other embodiments lignin fractions having relatively higher phenolic content were employed to increase scaffold density and mechanical reinforcement, and combinations or blends of lignin fractions may be employed to balance these properties.

In certain embodiments, optical stabilizers, chroma-mitigating additives, and/or color-modulating agents, including chlorophyll, chlorophyllin, carotenoids, flavonoids, or derivatives thereof, were incorporated to mitigate undesired chromogenic byproducts associated with enzyme-mediated oxidation of lignin and to maintain a cosmetically acceptable appearance of treated hair. Such additives may be selected to be compatible with enzyme activity, nanoparticle stability, and cosmetic safety. In further embodiments, additional cosmetic excipients, including fragrances, conditioning agents, botanical extracts, oils, esters, silicones, polymers, humectants, thickeners, preservatives, and/or surfactants, may be incorporated into the formulation without substantially interfering with nanoparticle integrity, enzyme activity, or scaffold formation.

Qualitative and/or semi-quantitative evaluations of treated hair fibers indicated improved glide and reduced friction during combing, brushing, detangling, and/or heat styling, as well as improved elasticity, curl cohesion after drying, and reduced strand fracture in highly coiled, curly, and/or damaged hair textures. Such performance observations are consistent with internal load distribution and mechanical reinforcement provided by formation of the intrafiber scaffold within the cortex, rather than surface coating or chemical crosslinking of keratin.

In certain embodiments, chromogenic byproducts associated with oxidative polymerization of lignin phenolic moieties are mitigated or suppressed to maintain cosmetic transparency or color neutrality. For example, optical stabilizers, chroma-mitigating agents, chelating agents, antioxidants, chlorophyll derivatives, chlorophyllin derivatives, or related cosmetic-compatible additives may be incorporated to reduce visible browning, yellowing, or discoloration associated with enzyme-mediated oxidation. Such additives may be selected to preserve enzyme activity and scaffold formation while maintaining a cosmetically acceptable appearance of treated hair fibers.

In certain embodiments, analytical and characterization methods may be employed to validate nanoparticle penetration, intrafiber localization, and scaffold formation, including, but not limited to, scanning electron microscopy (SEM), transmission electron microscopy (TEM), confocal or fluorescence microscopy (with optional labeling), atomic force microscopy (AFM), nanoindentation, Raman spectroscopy, Fourier-transform infrared (FTIR) spectroscopy, and/or other spectroscopic techniques to confirm oxidative polymerization signatures of lignin within the cortex. Mechanical performance may be further evaluated by single-fiber tensile testing, cyclic fatigue testing, and/or dynamic mechanical analysis (DMA) under varying humidity and environmental conditions.

It will be understood that the foregoing prototype embodiments, observations, and characterization techniques are exemplary only, and that other formulation variants, activation conditions, analytical methods, and performance metrics may be employed to assess and implement the disclosed compositions and methods without departing from the scope of the invention.

### DETAILED DESCRIPTION OF THE FIGURES

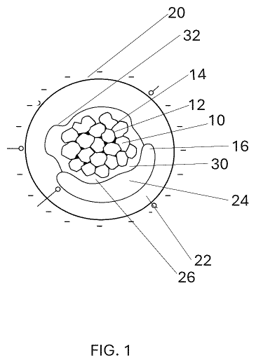

### FIG. 1—Nanoparticle Penetration into Hair Fiber

FIG. 1 schematically illustrates the delivery and penetration of composite lignin-starch nanoparticles (10) into a hair fiber (20). The nanoparticle (10) comprises a lignin phase (12) including phenolic/aromatic domains and a starch phase (14) forming a polysaccharide matrix. In certain embodiments, surface charge regions (16) are distributed on an exterior surface of the nanoparticle (10).

The hair fiber (20) includes a cuticle layer (22), a cortex region (24), and a medulla region (26). In the illustrated embodiment, composite lignin-starch nanoparticles (10) migrate along one or more penetration pathways (30) through cuticle interstices (32) of the cuticle layer (22) to localize within the cortex region (24). In certain embodiments, localization of the nanoparticles (10) occurs predominantly or exclusively within the cortex region (24) without substantial penetration into the medulla region (26). The figure is schematic and not to scale.

In certain embodiments, preferential scaffold formation at Z-entry junctions reduces localized tensile and torsional stress concentrations at condensed coil or bend regions during elongation and styling. Reinforcement of such regions redistributes mechanical load away from high-curvature zones, thereby reducing fracture initiation, micro-buckling, and fatigue-related damage during repeated mechanical deformation of coiled and highly curved hair fibers.

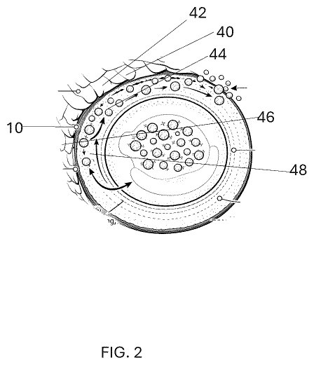

### FIG. 2—Enzyme-Activated Polymerization of Nanoparticles

FIG. 2 schematically illustrates enzyme-activated polymerization/enzyme-mediated polymerization of composite lignin-starch nanoparticles (10) following localization within a cortex region (24) of a hair fiber (20). In the illustrated embodiment, a phenol oxidase enzyme, such as laccase enzyme (40) interacts with phenolic moieties of the lignin phase (12) to generate radical formation sites (42) on the nanoparticles (10).

Inter-nanoparticle polymer bridges (44) form between adjacent nanoparticles (10) within a polymerization reaction zone (46), thereby initiating formation of a polymerized intrafiber scaffold (48). In certain embodiments, polymerization is configured to occur after intrafiber localization of the nanoparticles (10). The figure is schematic and not to scale.

In certain embodiments, enzyme-mediated polymerization and inter-nanoparticle crosslink formation may preferentially initiate after nanoparticle localization within regions of the cortex that experience higher local strain, curvature, or microenvironmental variation (e.g., moisture or oxygen gradients), such that scaffold formation is biased toward mechanically stressed regions of the hair fiber.

In certain embodiments, enzyme activity and polymerization kinetics may be regulated by local oxygen availability, moisture content, and temperature within the cortex, such that inter-nanoparticle crosslink formation occurs after cortical penetration rather than at the cuticle surface.

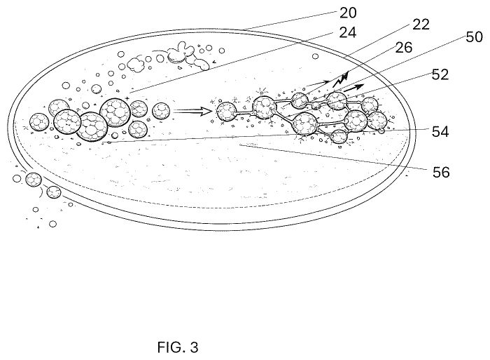

### FIG. 3—Polymerized Intrafiber Scaffold Within Cortex

FIG. 3 schematically illustrates a cross-sectional view of a hair fiber (20) showing formation of a polymerized intrafiber scaffold (50) within a cortex region (24). The hair fiber (20) includes a cuticle layer (22) surrounding the cortex region (24) and a medulla region (26) disposed radially inward of the cortex region (24).

The polymerized intrafiber scaffold (50) comprises an interconnected network formed from polymerized lignin-starch nanoparticles (10) and is physically distinct from keratin microfibrils (52) of the hair fiber (20). In certain embodiments, the polymerized intrafiber scaffold (50) remains confined to the cortex region (24) and is substantially excluded from the medulla region (26), which may be separated by an exclusion boundary (56). In some embodiments, a minor fraction of non-polymerized nanoparticles (54) may remain dispersed within the cortex region (24) following polymerization. The figure is schematic and not to scale.

In certain embodiments, the polymerized intrafiber scaffold (50) defines a percolative network topology that extends across a plurality of keratin microfibrils (52) within the cortex (24) and occupies interstitial spaces between adjacent keratin microfibrils. Such a percolative network distributes mechanical load across multiple microfibrils and provides internal reinforcement of the cortex without chemical modification of keratin.

In certain embodiments, the polymerized intrafiber scaffold (50) forms a three-dimensional nanoscale network topology within the cortex (24) that is physically distinct from the keratin microfibrils (52), with the scaffold and keratin constituting separate polymeric phases that may be in interfacial contact without covalent crosslinking to keratin.

In certain embodiments, localization and polymerization of the composite nanoparticles (10) and formation of the intrafiber scaffold (50) occur predominantly within the cortex (24), with the medulla (26) remaining substantially free of scaffold material, thereby confining structural reinforcement to the load-bearing region of the hair fiber (20).

In certain embodiments, intrafiber scaffold formation may be locally concentrated in regions of higher fiber curvature within the cortex (24), such that internal reinforcement aligns with the natural curvature or torsional geometry of the hair fiber, thereby contributing to stabilization of curl pattern under mechanical or humidity-induced stress.

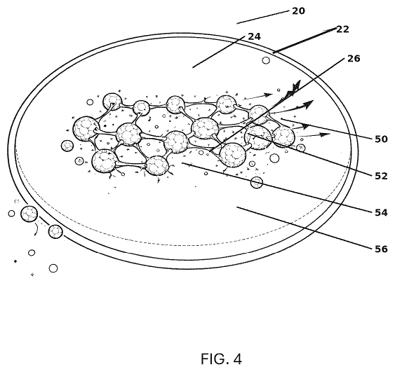

### FIG. 4—Reinforced Hair Fiber Cross-Section

FIG. 4 schematically illustrates a cross-sectional view of a hair fiber (20) after formation of a polymerized intrafiber scaffold (50) within a cortex region (24). The polymerized intrafiber scaffold (50) forms an interconnected internal network derived from lignin-starch nanoparticles (10) and is physically distinct from keratin microfibrils (52).

In certain embodiments, the intrafiber scaffold (50) reinforces the cortex region (24) and contributes to enhanced mechanical response of the hair fiber (20) under applied tensile, bending, or torsional loading relative to an untreated hair fiber. In certain embodiments, the scaffold (50) remains confined to the cortex region (24) without penetrating into the medulla region (26). The figure is schematic and not to scale.

In certain embodiments, the disclosed compositions and methods result in structural modification predominantly or exclusively within the cortex of the hair fiber, with minimal or no structural modification within the medulla and without permanent modification of the cuticle structure. The intrafiber scaffold is configured to reinforce internal architecture while preserving cuticle integrity and native keratin chemistry. Such cortex-selective reinforcement enables internal strengthening without surface coating, protein crosslinking, or chemical alteration of keratin disulfide bonds.

In certain embodiments, the polymerized intrafiber scaffold (50) modifies the mechanical response of the hair fiber (20) under applied tensile or bending loads by redistributing mechanical stress across multiple keratin microfibrils within the cortex (24). Such redistribution reduces localized stress concentrations that would otherwise promote microfracture or premature failure of the keratin microfibrils.

In certain embodiments, the enhanced mechanical response illustrated in FIG. 4 arises from physical reinforcement provided by the intrafiber scaffold (50), rather than from chemical modification of keratin, rearrangement of disulfide bonds, or formation of covalent crosslinks with keratin protein. The scaffold acts as a mechanically supportive internal framework within the cortex.

In certain embodiments, the presence of the intrafiber scaffold (50) increases tensile strength, elongation before break, and resistance to fracture relative to untreated hair fibers by providing internal load-bearing pathways that accommodate elastic deformation and reduce microfibril slippage under strain.

In certain embodiments, the reinforced mechanical response persists under cyclic loading and humidity variation, such that the intrafiber scaffold (50) contributes to retention of mechanical integrity and curl stability after repeated hydration-dehydration cycles.

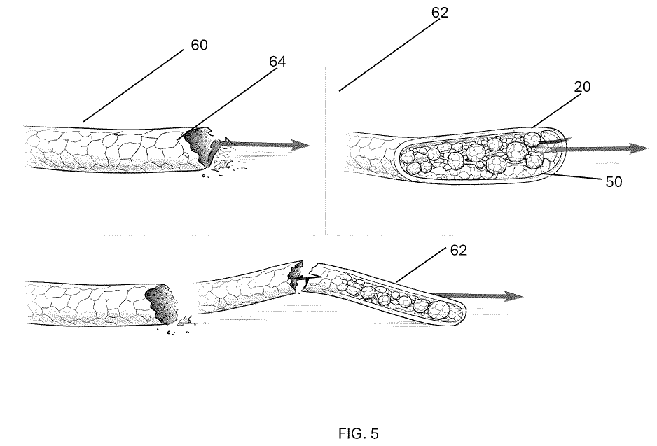

### FIG. 5—Comparative Mechanical Response of Treated vs. Untreated Hair Fibers

FIG. 5 schematically illustrates comparative mechanical responses of an untreated hair fiber (60) and a treated hair fiber (20) containing a polymerized intrafiber scaffold (50) within a cortex region (24) when subjected to an applied tensile force (62).

In the illustrated embodiment, the untreated hair fiber (60) exhibits lower tensile strength and reduced elongation under applied force (62) and may undergo failure at a failure region (64). By contrast, the treated hair fiber (20) exhibits enhanced elongation under strain and increased resistance to failure due to reinforcement provided by the polymerized intrafiber scaffold (50) formed within the cortex region (24).

In certain embodiments, treated hair fibers exhibit increases in tensile strength (e.g., about 5-28%), elongation before break (e.g., about 40-70%), and/or flexibility (e.g., about 8-30%) relative to untreated hair fibers under comparable test conditions. The figure is schematic and not to scale.

In certain embodiments, the comparative mechanical response illustrated in FIG. 5 demonstrates that hair fibers containing the polymerized intrafiber scaffold (50) exhibit increased elongation under applied tensile force and delayed onset of fracture relative to untreated hair fibers (60) lacking the scaffold. This comparative response highlights the contribution of internal scaffold formation to mechanical reinforcement beyond surface conditioning effects.

In certain embodiments, the magnitude of improvement in tensile strength and elongation observed in scaffold-containing hair fibers exceeds that achievable by conventional surface-deposited conditioners or bond-building treatments, indicating that internal intrafiber reinforcement yields mechanical benefits not predicted by surface-only or keratin-bond-modifying approaches.

In certain embodiments, formation of the intrafiber scaffold (50) alters the failure mode of the hair fiber under tensile loading, shifting fracture initiation away from localized microfibril failure zones and toward more distributed deformation across the cortex, thereby increasing resistance to catastrophic breakage.

In certain embodiments, the treated hair fiber (20) maintains enhanced mechanical performance under elevated humidity conditions, with reduced deformation and frizz formation relative to untreated hair fibers, consistent with internal load-bearing reinforcement provided by the polymerized intrafiber scaffold (50).

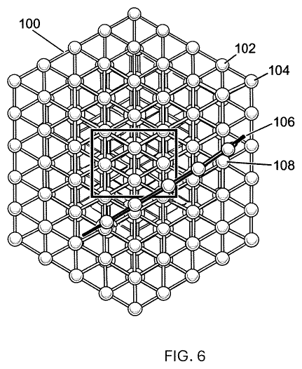

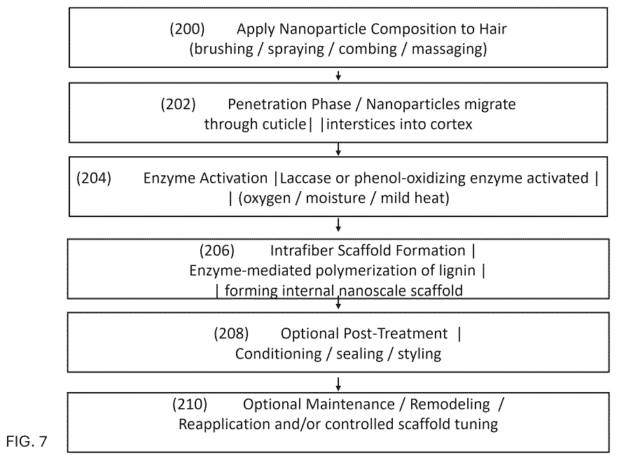

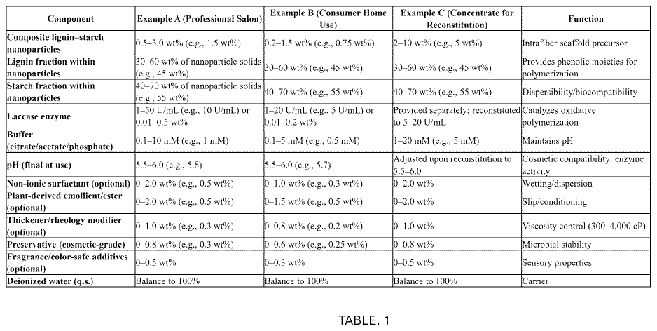

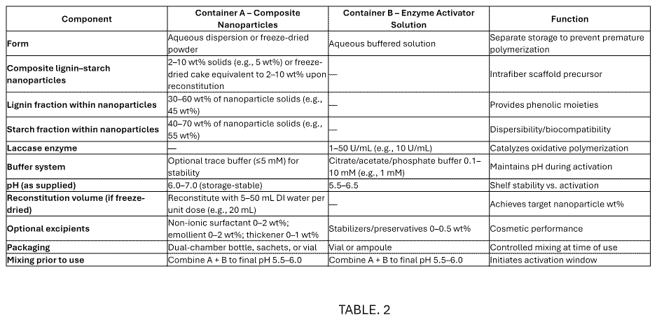

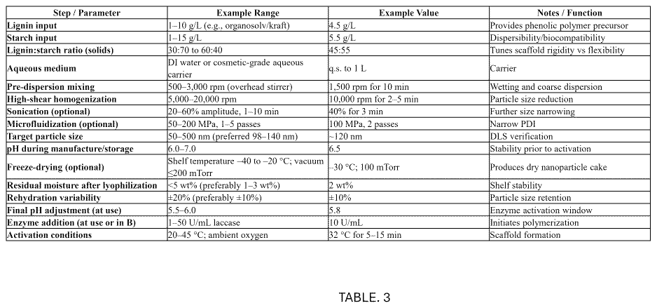

### FIG. 6—Conceptual Polymer Network Topology (Nanoscale Scaffold)

FIG. 6 schematically illustrates a conceptual representation of a three-dimensional scaffold lattice (100) corresponding to a polymer network topology formed by polymerized lignin-starch nanoparticles (10) within a cortex region (24) of a hair fiber (20).

In the illustrated embodiment, the scaffold lattice (100) comprises a plurality of interconnected nodes (102) coupled by connecting struts or bridges (104), which together define repeating network motifs (106) forming a porous, three-dimensional nanoscale scaffold (50). The nodes (102) may correspond to polymerized nanoparticle junctions and the connecting struts (104) may correspond to inter-nanoparticle polymer bridges formed during enzyme-activated polymerization/enzyme-mediated polymerization.

In certain embodiments, the scaffold lattice (100) defines one or more load-bearing pathways (108) configured to transmit mechanical loads through the nanoscale scaffold (50). The representation of the scaffold lattice (100) is schematic and not to scale and is intended to illustrate network connectivity and topology rather than any literal or limiting geometric lattice structure. The nanoscale scaffold (50) is physically distinct from keratin microfibrils (52) and is configured to provide internal mechanical reinforcement within the cortex region (24).

In certain embodiments, the scaffold lattice may comprise irregular, non-periodic, percolating, clustered, and/or heterogeneous network architectures rather than ordered lattices.

In certain embodiments, the intrafiber scaffold formed by enzyme-activated polymerization/enzyme-mediated polymerization of lignin-starch composite nanoparticles adopts a three-dimensional network topology that may be conceptually described as fullerene-inspired or “buckyball-like” in its connectivity, symmetry, and load-bearing pathways, without requiring or incorporating carbon nanomaterials, graphene, fullerenes, carbon nanotubes, or inorganic nanostructures. In such embodiments, the term “fullerene-inspired” refers to network topology and connectivity motifs, including closed or semi-closed polyhedral arrangements, nodal interconnectivity, and distributed load-bearing pathways, rather than chemical composition. The scaffold network is formed from biodegradable polymeric materials and is configured to provide mechanical reinforcement within the cortex while remaining compatible with cosmetic use and biological substrates.

References to spherical, cage-like, or fullerene-inspired nanostructures herein are conceptual and relate to three-dimensional network topology and mechanical load distribution, rather than literal carbon fullerene chemistry.

References to spherical, cage-like, fullerene-inspired, lattice-like, or polyhedral architectures are conceptual and non-limiting, and are intended to describe network topology and mechanical connectivity rather than any specific molecular or carbon-based nanostructure.

In certain embodiments, the network topology illustrated in FIG. 6 is schematic and representative of one of many possible three-dimensional intrafiber scaffold topologies. The illustrated nodes and connecting struts are intended to represent inter-nanoparticle crosslink junctions and polymerized lignin-rich domains, and do not limit the scaffold to any particular geometric lattice, periodicity, symmetry, or crystalline structure.

In certain embodiments, the polymerized intrafiber scaffold defines a percolative network topology within the cortex, such that interconnected scaffold pathways extend across multiple keratin microfibrils and occupy interstitial spaces therebetween, thereby establishing load-bearing pathways that distribute mechanical stress through the cortex.

In certain embodiments, the intrafiber scaffold comprises a heterogeneous and non-uniform network architecture, including regions of higher crosslink density and regions of lower crosslink density, such that mechanical reinforcement is spatially distributed in a non-periodic manner within the cortex.

In certain embodiments, the polymerized intrafiber scaffold depicted schematically in FIG. 6 remains physically distinct from keratin microfibrils, such that the scaffold and keratin constitute separate polymeric phases that may be in interfacial contact without covalent bonding to keratin protein chains.

In certain embodiments, network connectivity and scaffold density may be locally enhanced in regions of higher fiber curvature or torsional stress within the cortex, such that the intrafiber scaffold provides preferential reinforcement aligned with the intrinsic curl geometry of the hair fiber.

### FIG. 7—Method Flow Diagram for Intrafiber Scaffold Formation

FIG. 7 illustrates a representative, non-limiting flow diagram of a method for forming a polymerized intrafiber scaffold within a hair fiber cortex using composite lignin-starch nanoparticles and an enzyme system. The illustrated method includes optional and/or alternative steps, and the order of steps may be modified, repeated, combined, and/or omitted without departing from the scope of the invention.

This flow diagram illustrates a representative method for intrafiber scaffold formation, including application of composite lignin-starch nanoparticles (200), penetration into the cortex (202), enzyme activation (204), enzyme-mediated polymerization/enzyme-activated polymerization and scaffold formation (206), optional post-treatment conditioning or sealing (208), and optional maintenance or remodeling treatments (210). The illustrated steps are non-limiting and may be reordered, repeated, combined, or omitted without departing from the scope of the invention.

In a delivery step (200), a composition comprising composite lignin-starch nanoparticles is applied to hair fibers. The composition may be provided in a single formulation or as part of a multi-part kit and may be delivered by brushing, spraying, combing, massaging, and/or other cosmetic application techniques.

In a penetration phase (202), the nanoparticles migrate through cuticle interstices into the cortex region of the hair fiber. In certain embodiments, penetration is facilitated by moisture, mild thermal exposure, mechanical handling, surfactants, penetration facilitators, and/or formulation viscosity control. In some embodiments, nanoparticles are configured to preferentially localize within the cortex while remaining substantially excluded from the medulla.

In an activation step (204), an enzyme system, such as laccase or other phenol-oxidizing enzymes, is applied and/or activated after nanoparticle penetration. Activation may be triggered by oxygen exposure, moisture, temperature elevation, removal of enzyme inhibitors, and/or mixing of separated formulation components.

In a polymerization step (206), enzyme-mediated oxidative coupling of lignin phenolic moieties occurs within the cortex, resulting in inter-nanoparticle association and/or covalent polymer bridging to form a three-dimensional intrafiber scaffold physically distinct from keratin microfibrils.

In a conditioning and/or sealing step (208), optional post-treatment compositions, such as conditioners, sealants, oils, emulsions, silicones, and/or protective films, may be applied to the hair surface to enhance cosmetic feel, moisture retention, and/or environmental resistance without disrupting the intrafiber scaffold.

In an optional maintenance or remodeling step (210), subsequent treatments may be performed to increase, restore, modulate, or partially disassemble scaffold density over time. In certain embodiments, scaffold density may be reduced or remodeled using solvent systems configured to disrupt non-covalent interactions within the scaffold without cleaving keratin disulfide bonds.

The method illustrated in FIG. 7 may be implemented as a single-step treatment, a multi-step professional salon protocol, a consumer home-use system, or a repeated maintenance regimen. In certain embodiments, individual steps may be omitted, repeated, reordered, and/or combined, provided that formation of an intrafiber scaffold within the cortex is achieved.

In certain embodiments, the method steps illustrated in FIG. 7 are representative and non-limiting, and the steps may be performed in different orders, in parallel, or with one or more steps added, omitted, repeated, or combined, without departing from the scope of the disclosed methods, provided that composite nanoparticle penetration into the cortex occurs prior to substantial intrafiber polymerization.

In certain embodiments, enzyme activity is suppressed, inhibited, or kinetically limited during an initial penetration phase to permit migration of the composite nanoparticles into the cortex prior to initiation of oxidative crosslink formation, and is subsequently activated within the cortex by exposure to oxygen, moisture, temperature elevation, pH adjustment, or combinations thereof.

In certain embodiments, the method comprises a multi-stage application protocol, including a first stage in which the composite nanoparticles are delivered to the hair fiber, and a second stage in which the phenol-oxidizing enzyme is applied or activated to induce intrafiber scaffold formation, with optional intermediate rinsing, conditioning, or dwell periods between stages.

In certain embodiments, polymerization kinetics are controlled by regulating one or more of oxygen availability, temperature, moisture content, enzyme concentration, nanoparticle concentration, and dwell time, such that scaffold density and spatial distribution within the cortex may be tuned based on hair type, curl pattern, or desired mechanical outcome.

In certain embodiments, the method is configured such that substantial polymerization and scaffold formation occur within the cortex after nanoparticle penetration, while polymerization at the cuticle surface is minimized or suppressed to avoid formation of surface films or coatings.

### Table 1. Representative Example Formulations

In certain embodiments, the compositions described herein include representative example formulations comprising composite lignin-starch nanoparticles, an enzyme system (e.g., laccase and/or other phenol-oxidizing enzymes), buffering agents, and cosmetically acceptable carriers. Table 1 provides representative, non-limiting examples of formulation compositions, ingredient classes, concentration ranges, pH values, and optional excipients suitable for intrafiber scaffold formation within the cortex of a hair fiber. The examples set forth in Table 1 are provided to illustrate the enablement and practical implementation of the disclosed compositions and do not limit the scope of formulation compositions encompassed by the invention.

In certain embodiments, representative formulations include composite lignin-starch nanoparticles in an amount of about 0.01 wt % to about 5 wt % (for example, about 0.1 wt %, 0.2 wt %, 0.5 wt %, 1 wt %, 2 wt %, or 3 wt %), a phenol oxidase enzyme, such as laccase or other phenol-oxidizing enzymes at activities of about 0.1 to about 100 U/mL, buffering agents configured to maintain formulation pH in a range of about 5.0 to about 6.5 (including about 5.5 to about 6.0), and cosmetically acceptable aqueous and/or emulsion-based carriers. Optional excipients may include, without limitation, surfactants, emulsifiers, humectants, emollients, thickeners, rheology modifiers, preservatives, antioxidants, fragrances, chelating agents, and/or color-safe additives, provided that such excipients do not substantially interfere with nanoparticle penetration, enzyme stability, and/or scaffold formation.

Table 1 sets forth representative, non-limiting example formulations of composite lignin-starch nanoparticle compositions suitable for intrafiber scaffold formation. The specific ingredient classes, concentration ranges, and formulation parameters illustrated in Table 1 are provided for purposes of enablement and illustration, and are not intended to limit the scope of the disclosed compositions, which may include other concentrations, ratios, excipients, and formulation formats consistent with the principles described herein.

In certain embodiments, the relative amounts of composite lignin-starch nanoparticles, enzyme system, buffers, and cosmetically acceptable carriers may be varied across the ranges illustrated in Table 1 to tailor penetration behavior, polymerization kinetics, scaffold density, and mechanical reinforcement outcomes based on hair type, fiber diameter, porosity, curl pattern, or desired cosmetic effect.

In certain embodiments, the lignin phase represented in Table 1 includes unmodified lignin, chemically functionalized lignin (e.g., carboxyl-functionalized lignin), or combinations thereof, and the starch phase includes native or modified starches, such that the composite nanoparticles may be chemically tailored to adjust enzyme reactivity, scaffold formation kinetics, and cosmetic transparency.

In certain embodiments, the formulation pH values illustrated in Table 1 represent cosmetically compatible pH ranges suitable for enzyme stability and activation, and buffering systems may be selected to maintain pH during penetration and polymerization phases without inducing cuticle swelling or disulfide bond cleavage.

In certain embodiments, formulations illustrated in Table 1 may be prepared in liquid form, concentrate form, or freeze-dried form for reconstitution prior to use, and stabilizers, preservatives, antioxidants, or packaging configurations may be employed to maintain nanoparticle dispersion stability and enzyme activity during storage.

### Table 2. Representative Two-Part Kit Configurations and Reconstitution Parameters

In certain embodiments, the compositions described herein are provided as multi-part kits to improve shelf stability, control timing of enzyme activation, and facilitate user preparation immediately prior to application. Table 2 illustrates representative, non-limiting kit formats in which composite lignin-starch nanoparticles are provided in a first container (optionally in freeze-dried, spray-dried, or concentrated liquid form) and an enzyme activator solution is provided in a second container, together with example concentration ranges, buffer systems, pH ranges, packaging formats, and reconstitution volumes suitable for forming an enzyme-activated intrafiber scaffold upon mixing and application to hair.

Separating the composite nanoparticles from the enzyme system may reduce or prevent premature polymerization during storage and transport. The values and configurations set forth in Table 2 are illustrative and may be adjusted based on formulation objectives, hair type, packaging constraints, shelf-life requirements, regulatory considerations, and desired treatment intensity. In certain embodiments, the kit may further include pretreatment compositions, post-treatment sealants, conditioners, applicators, mixing vessels, or instructions for staged application, without departing from the scope of the invention.

Table 2 sets forth representative, non-limiting examples of multi-part cosmetic kit configurations in which composite lignin-starch nanoparticles and an enzyme system are provided in physically separated containers prior to use. The specific concentrations, packaging formats, reconstitution volumes, and component combinations illustrated in Table 2 are provided for purposes of enablement and illustration and are not intended to limit the scope of the disclosed kit embodiments.

In certain embodiments, physical separation of the composite nanoparticles and the enzyme system prior to use inhibits premature oxidative crosslink formation during storage and transport and permits controlled initiation of polymerization only after intrafiber localization within the cortex following application to hair.

In certain embodiments, the kit components illustrated in Table 2 may be provided in dual-chamber packages, break-seal vials, coupled syringes, sequential pump dispensers, or other multi-compartment packaging configurations that permit controlled mixing of components immediately prior to or during application. In further embodiments, applicators may be oxygen-permeable, oxygen-restrictive, or selectively vented to regulate oxygen exposure during enzyme activation.

In certain embodiments, composite lignin-starch nanoparticles may be provided in a dry or freeze-dried form in a first container and reconstituted with an aqueous buffer, activator solution, or cosmetically acceptable carrier provided in a second container prior to application. Reconstitution volumes, mixing ratios, and order of application may be varied to tailor penetration kinetics, scaffold density, and treatment intensity.

In certain embodiments, the kit may further include optional pretreatment compositions, primers, penetration-enhancing compositions, post-treatment conditioners, sealants, neutralizers, oxygen modulators, thermal caps, applicators, or instructional materials, provided that such components do not substantially inhibit nanoparticle penetration or enzyme-mediated oxidative crosslink formation within the cortex.

### Table 3. Representative Manufacturing Batch Recipe and Processing Parameters

Table 3 sets forth representative, non-limiting examples of material inputs, processing ranges (including aqueous dispersion, controlled agitation, homogenization, sonication, microfluidization, or combinations thereof), stabilization steps such as freeze-drying and/or spray-drying, residual moisture targets, and final pH adjustment and enzyme activation conditions that may be used to produce composite lignin-starch nanoparticles and prepare formulations for intrafiber scaffold formation.

These examples illustrate one of many suitable manufacturing pathways for producing the disclosed compositions and are provided to demonstrate enablement. Variations in materials, lignin and starch source, weight ratios, equipment type, batch size, order of processing steps, drying methods, and process conditions may be employed to tailor nanoparticle size, morphology, stability, enzyme accessibility, and performance characteristics without departing from the scope of the invention.

Table 3 sets forth representative, non-limiting examples of batch-scale manufacturing recipes and processing parameters suitable for producing composite lignin -starch nanoparticles for cosmetic hair treatment applications. The specific material inputs, concentration ranges, equipment types, processing conditions, and stabilization steps illustrated in Table 3 are provided for purposes of enablement and illustration and do not limit the scope of manufacturing methods encompassed by the invention.

In certain embodiments, the processing parameters illustrated in Table 3, including agitation rate, homogenization pressure, sonication power, microfluidization cycles, temperature, and solids loading, are adjusted to control nanoparticle size distribution and surface properties, such that composite nanoparticles having average diameters within a range of about 50 nm to about 500 nm, including subranges of about 50-200 nm, about 80-120 nm, or about 100-150 nm, are produced.

In certain embodiments, stabilization steps illustrated in Table 3, including freeze-drying, spray-drying, or lyophilization, yield composite nanoparticles with residual moisture contents of less than about 5 wt %, optionally less than about 3 wt %, to improve shelf stability, transport robustness, and reconstitution consistency. Cryoprotectants, bulking agents, or stabilizers may be included to preserve nanoparticle morphology upon rehydration.

In certain embodiments, the processing conditions and excipients illustrated in Table 3 are selected to preserve compatibility between the composite nanoparticles and phenol-oxidizing enzymes used in the disclosed compositions, such that nanoparticle integrity and enzyme activity remain substantially intact upon mixing and during use. Manufacturing conditions may be selected to minimize premature oxidative crosslink formation prior to intrafiber localization.

In certain embodiments, the batch recipes illustrated in Table 3 may be implemented at laboratory scale, pilot scale, or commercial manufacturing scale, and processing parameters may be proportionally adjusted for batch size, equipment configuration, or throughput without departing from the scope of the invention. Continuous or semi-continuous processing may be employed in further embodiments.

In certain embodiments, composite nanoparticles produced according to the manufacturing parameters illustrated in Table 3 exhibit reconstitution behavior in aqueous cosmetic carriers with average particle size deviation of less than about ±10% relative to pre-drying values, supporting consistent penetration and scaffold formation performance across manufacturing lots.

This written description uses examples to disclose representative embodiments of the invention, including best-mode embodiments, and to enable any person skilled in the art to practice the invention, including making and using compositions, kits, and systems and performing the disclosed methods. The patentable scope of the invention is defined by the claims and may include other embodiments and variations that occur to those skilled in the art. Such other embodiments are intended to be within the scope of the claims if they include structural elements or method steps that do not differ materially from the literal language of the claims, or if they include equivalent elements or steps with insubstantial differences from the literal language of the claims.

It is to be understood that the above description is intended to be illustrative and not restrictive. For example, the above-described embodiments (and/or aspects thereof) may be used in combination with each other. In addition, many modifications may be made to adapt a particular situation, method, system device, or material to the teachings of the various embodiments of the invention without departing from their scope. While the particulars and details described herein are intended to define the parameters of the various embodiments of the invention, the embodiments are by no means limiting and are exemplary embodiments. Many other embodiments will be apparent to those of skill in the art upon reviewing the above description. The scope of the various embodiments of the invention should, therefore, be determined with reference to the appended claims, along with the full scope of equivalents to which such claims are entitled.

1. A hair treatment composition comprising:
(a) composite nanoparticles each comprising a carboxyl-functionalized lignin phase and a starch phase configured to self-assemble into spherical nanostructures having average diameters of about 50-200 nm;
(b) a laccase enzyme present in an amount effective to catalyze oxidative crosslink formation of carboxyl moieties after intrafiber localization; and
(c) a cosmetically acceptable aqueous carrier;
wherein the composition has a pH of 5.5-6.0, and forms within a hair cortex a polymerized lignin-based intrafiber scaffold that is physically distinct from keratin and formed without disulfide bond cleavage.

2. The composition of claim 1, wherein polymerization forms a percolating nanoscale intrafiber scaffold within the cortex occupying interstitial spaces between keratin microfibrils.

3. The composition of claim 1, wherein the composite nanoparticles penetrate through a coil-bend inflection region (“Z-entry junction”) exhibiting a localized angular change in a curl path that produces a transient porous junction, facilitating deeper nanoparticle entry, having a capacity to extend across a plurality of keratin microfibrils within the cortex.

4. The composition of claim 3, wherein Z-entry penetration reduces tensile stress on a condensed-coil region during elongation by distributing mechanical load away from torsion-dense zones.

5. The composition of claim 1, wherein the composite nanoparticles comprise lignin-rich functional domains stabilized by starch-rich supportive domains within a unified composite morphology, so that a lignin-rich domains serve as the polymerizable phase upon laccase activation.

6. The composition of claim 1, wherein the nanoparticles exhibit a zeta potential promoting electrostatic association with hair fibers.

7. The composition of claim 1, wherein scaffold density within the cortex is adjustable by nanoparticle concentration.

8. The composition of claim 1, wherein the nanoparticles have diameters within 50-150 nm.

9. The composition of claim 1, wherein the nanoparticles have diameters within 80-120 nm.

10. The composition of claim 1, wherein the lignin phase comprises functionalized lignin derived from a lignin source selected from the group consisting of organosolv lignin, kraft lignin, soda lignin, and combinations thereof, the lignin comprising carboxyl-functional groups.

11. The composition of claim 1, wherein the starch phase is derived from a starch source selected from the group consisting of corn starch, potato starch, tapioca starch, rice starch, and combinations thereof.

12. The composition of claim 1, wherein the lignin is sourced from upcycled biomass feedstocks.

13. The composition of claim 1, wherein the nanoparticles are provided in a freeze-dried form configured for reconstitution before use.

14. The composition of claim 1, wherein the composition is substantially free of alkali, and nanoparticle penetration into the cortex occurs primarily through nanoparticle-scale cuticle interstices and the coil-bend inflection (“Z-entry”) region, rather than chemically induced cuticle lifting.

15. The composition of claim 1, wherein the composition is configured to preserve cuticle integrity during treatment.

16. A method of treating hair comprising:
applying to hair a composition comprising composite lignin-starch nanoparticles and a laccase enzyme at a pH of 5.5 to 6.0;
permitting the nanoparticles to migrate through cuticle interstices into the cortex;
and activating the laccase within the cortex to induce oxidative crosslink formation of carboxyl moieties of the lignin phase and establishment of a polymerized intrafiber scaffold physically distinct from keratin.

17. The method of claim 16, wherein nanoparticle migration includes entry through a coil-bend inflection region (“Z-entry junction”) exhibiting increased porosity due to angular torsion of the curl path.

18. The method of claim 16, wherein the polymerized scaffold forms a percolative network within interstitial spaces between keratin microfibrils.

19. A cosmetic hair treatment kit comprising:
a first container including composite lignin-starch nanoparticles;
a second container including a laccase enzyme in a cosmetically acceptable buffer;
and instructions directing cortical migration followed by enzymatic activation to form an intrafiber scaffold.

20. The kit of claim 19, further comprising an oxygen-permeable applicator configured to regulate oxygen exposure so that oxidative crosslink formation initiates within the cortex after nanoparticle penetration.
